(19) United States
(12) Patent Application Publication
(10) Pub. No.: US 2017/0360829 A1
(43) Pub. Date: Dec. 21, 2017

(54) TREATMENT OF VASCULAR ANOMALIES

(71) Applicant: GILLIES MCINDOE RESEARCH INSTITUTE, Newtown (NZ)

(72) Inventors: Swee Thong Tan, Wellington (NZ); Paul Frank Davis, Wellington (NZ); Tinte Itinteang, Wellington (NZ)

(21) Appl. No.: 15/444,033

(22) Filed: Feb. 27, 2017

Related U.S. Application Data
(60) Provisional application No. 62/287,657, filed on Jan. 27, 2016.

(51) Int. Cl. A61K 33/06; A61K 45/00; A61K 31/59

(52) U.S. Cl. CPC A61K 33/06; A61K 31/59; A61K 45/00

(57) ABSTRACT

The present invention provides a novel approach to the diagnosis, treatment and management of vascular anomalies by providing compositions and methods for the identification and specific targeting of the embryonic stem cell populations associated with vascular anomalies, by modulation of the Renin-Angiotensin System expressed by the stem cells.

### TECHNICAL FIELD

The present invention provides novel approaches to the treatment of vascular anomalies, for example, venous malformation and pyogenic granuloma. In particular, the present invention provides for the identification of embryonic stem cell populations expressing the renin-angiotensin system demonstrated here to be associated with vascular anomalies, and which embryonic stem cells provide a unique therapeutic target in the treatment of vascular anomalies such as venous malformation and pyogenic granuloma.

### BACKGROUND OF THE INVENTION

Vascular anomalies are classified by the International Society for the Study of Vascular Anomalies classification system into vascular tumors and vascular malformations.¹ Vascular malformations may affect arteries, veins, lymphatics and capillaries singly or in combinations, with venous malformation (VM) being the most common.¹ The biology of each condition may be quite different meaning that identification/diagnosis and associated treatment regimens with respect to different vascular anomalies may require vastly different approaches for their management.

Venous malformation affects 1.5% of the population² and is characterized by thin-walled ectatic veins lined by flat endothelial cells (EC) with deficient or absent smooth muscle cells (SMC).^{3,4} Venous malformation may affect any body sites and tissues⁴ commonly in the subcutaneous (SC) and less commonly in intramuscular (IM) locations.⁵ Although VM is present at birth, it may not become apparent until later in life.³⁻⁵ Its clinical presentation depends on the location and size of the lesion. Subcutaneous lesions present as compressible masses with a bluish hue, whereas IM lesions often present with a swelling and/or pain.⁴ Venous malformation may cause cosmetic concerns, and/or functional deficits such as obstructive sleep apnoea as in the case of oropharyngeal lesions.⁶ Venous malformation grows proportionately with the growth of the child, and may suddenly expand in response to hormonal changes or trauma, including incomplete surgical excision.³⁻⁵

1-2% of VM cases are familial caused by a TIE2 mutation.⁷ Half of the sporadic cases also have a TIE2 somatic mutation.⁷ These mutations have been shown to result in ligand-independent hyperphosphorylation of the TIE2 receptor.⁷ Vikkula et al.⁸ suggest that the TIE2 mutation in the endothelial cells (EC) in VM may reduce SMC ligand expression causing a local uncoupling between normal SMC recruitment and the proliferation of EC.

Treatment of VM includes sclerotherapy⁹, surgical excision¹⁰, laser therapy¹¹ and combined treatments.^{12,13} A review by van der Vleuten et al.¹⁴ assessing the effectiveness of various treatment modalities and sclerosing agents for VM shows that surgery was effective in 90% of cases, laser in 94%, ethanol sclerotherapy in 74%, gelified ethanol in 89%, bleomycin in 88%, polidocanol in 82%, sodium tetradecyl sulfate in 86%, and Ethibloc in 65% of cases.¹⁴ Complications of sclerotherapy include parasthesia, nerve palsy, blistering, skin necrosis, ulceration, deep vein thrombosis, pulmonary embolism, and cardiac arrest.^{9,15-20} Reported complication rates range from 10-30%, with major complications occurring in up to 5.6% of CaSeS.^{9,21-23}

Pyogenic granuloma (PG) is another type of vascular anomaly. It is also known as lobular capillary hemangioma, and is an acquired benign vascular tumor⁴⁹ affecting the skin and mucous membranes.⁵⁰ The term pyogenic granuloma is a misnomer as it had been erroneously attributed to bacterial and mycotic infection.⁵¹ However, it is neither granulomatous nor purulent. Histologically, PG is composed of proliferating microvessels organized into lobules embedded in a fibromyxoid stroma⁵²⁻⁵⁴ with each lobule consisting of a central feeder vessel surrounded by clusters of small capillaries with an overlying epithelium that is often atrophic, flattened or ulcerated.^{55,56}

Pyogenic granuloma is a relatively common skin disorder, comprising 0.5% of all skin nodules in children.⁵⁴ A sub-group termed, epulis gravidarum, occurs in 5% of women during pregnancy.⁵⁷ Pyogenic granuloma most commonly affects the skin with approximately 12% involving the mucosa.^{54,58} Cutaneous lesions occur most commonly on the trunk, the extremities, and the head and neck area, in decreasing frequency.⁵⁸ There is no difference in the overall incidence of PG between genders. However, females are 2.5 times more likely to develop mucosal lesions compared with males.^{54,58,59} Pyogenic granuloma usually presents as a 1-2 cm, friable, red to purple pedunculated or sessile lesion that typically grows rapidly, often with repeated profuse bleeding following minor trauma. However, it may also grow to giant proportions.⁶⁰ Surgical excision remains the main treatment modality and is associated with a recurrence rate of 2.9%. Other treatment options include cryotherapy.⁶¹ Shave-excision and/or pulsed-dye laser therapy have been shown to be effective treatment modalities for PG affecting cosmetically sensitive areas.⁶²

Human ESCs are stem cells derived from the inner cell mass of the pre-implantation blastocyst that possess the capacity to form all three germ layers: endoderm, ectoderm and mesoderm.^{63,64} Since their first isolation in 1998,⁶³ over 300 human ESC lines have been developed.⁶⁵ Characterization of these cell lines has led to the discovery of a number of transcription factors and cell surface receptors including OCT4,⁶⁶ NANOG,⁶⁷ SOX2⁶⁸ and STAT3⁶⁹ that have been used to identify a ESC phenotype. OCT4, SOX2 and NANOG are transcription factors essential for maintaining pluripotency of ESC.⁷⁰ OCT4, a POU family transcription factor expressed on early pluripotent ESC, is involved in the maintenance of pluripotency through interactions with SOX2.⁶⁶ As part of the sex determining region Y family of transcription factors, SOX2 has been shown to play key roles in many stages of mammalian development, with a critical role in the maintenance of embryonic and neural stem cells.⁷¹⁻⁷³ OCT4 and SOX2 work synergistically to regulate the downstream NANOG, a homeobox containing transcription factor, implicated in self-renewal of undifferentiated ESCs through the activation of the Rex1 promoter region.⁷⁴ STAT3 directly regulates the actions of OCT4 by preventing the differentiation of cells into more mature lineages.⁷⁵ Addition of exogenous NANOG, OCT4 and SOX2 into fully differentiated adult cells has been shown to induce pluripotency, leading to the creation of induced pluripotent stem cells (iPSC).⁷⁶

The recent description of hemangioblastic blood islands within PG,^{56,77,78} implying the presence of a hematopoietic stem cell population,⁷⁹ led Applicants to investigate the expression of the more upstream markers of ESC, OCT4, SOX2, pSTAT3 and NANOG in pyogenic granuloma and venous malformation, as demonstrated within proliferating infantile hemangioma (IH).⁸⁰

The present invention is concerned methods for the identification and specific targeting of the embryonic stem cell populations associated with vascular anomalies, by modulation of the Renin-Angiotensin System expressed by the stem cells.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or embodiments identified in this Summary of the Invention, which is included for purposes of illustration only and not restriction.

The present invention is concerned with novel approaches to the treatment and management of vascular anomalies, including venous malformation and pyogenic granuloma, by specifically targeting stem cell populations which, for the first time, have been demonstrated by the Applicants to be associated with vascular anomalies. Accordingly, identification of these embryonic stem populations provides a novel approach to the management and treatment of vascular anomalies, as well as in prognostic, diagnostic and follow-up applications. In addition, Applicants have surprisingly demonstrated that these embryonic stem cells express markers associated with key regulatory systems including, for example, the Renin-Angiotensin System (RAS) including the Pro/Renin Receptor System (PRRS) and the associated bypass pathways. This insight provides a novel target and unique therapeutic opportunity in the management and treatment of vascular anomalies by employing established and/or novel drugs that specifically target these regulatory pathways in an attempt to eradicate, or arrest growth, proliferation and/or differentiation of embryonic stem cell populations which play a biological role in the pathology of these conditions.

Accordingly, in one aspect of the present invention there is provided a method for preventing, treating, or managing vascular anomalies in a patient in need thereof, the method comprising administering a therapeutic agent to the patient in an amount sufficient to selectively eradicate, or inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System.

In yet another aspect of the present invention there is provided a method for preventing, treating, or managing vascular anomalies in a patient in need thereof, the method comprising administering a therapeutic agent to the patient in an amount sufficient to selectively eradicate, or inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System, and wherein vascular anomalies is selected from the group consisting of venous malformation, pyogenic granuloma, infantile hemangioma, lymphatic malformation, capillary malformation, arterial malformation, arterio-venous malformation, lymphatico-venous malformation, capillary-venous malformation, capillary-lymphatico-venous malformation, kaposiform hemangioendothelioma, tufted angioma, hepatic hemangioendothelioma and verrucous hemangioma.

In another aspect of the present invention there is provided a method for preventing, treating, or managing vascular anomalies in a patient in need thereof, the method comprising administering a therapeutic agent to the patient in an amount sufficient to selectively eradicate, or inhibit the growth, proliferation and/or differentiation of embryonic stem cells associated with vascular anomalies, wherein the embryonic stem cells are characterized by (i) the expression of one or more stem cell biomarkers selected from the group consisting of Cripto, ABCG2, Alkaline Phosphatase/ALPL, CD9, FGF-4, GDF-3, Integrin alpha 6/CD49f, Integrin beta 1/CD29, NANOG, OCT-3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf, L1TD1, and (ii) the expression of one or more biomarkers associated with the Renin-Angiotensin System.

In yet another aspect of the present invention there is provided a method for preventing, treating, or managing vascular anomalies in a patient in need thereof, the method comprising administering a therapeutic agent to the patient in an amount sufficient to selectively eradicate, or inhibit the growth, proliferation and/or differentiation of embryonic stem cells associated with vascular anomalies, wherein the embryonic stem cells are characterized by (i) the expression of one or more embryonic stem cell biomarker selected from the group consisting of OCT4, SOX2, NANOG and PSTAT3, and (ii) the expression of one or more biomarkers associated with the Renin-Angiotensin System selected from the group consisting of Renin Receptor (RR), a soluble form of Angiotensin Converting Enzyme (ACE), Angiotensin II Receptor 1, Angiotensin II Receptor 2, and a soluble form of the Renin Receptor (sRR).

In yet a further aspect of the present invention there is provided a method for preventing, treating, or managing vascular anomalies in a patient in need thereof, the method comprising administering a therapeutic agent(s) to the patient in an amount sufficient to selectively eradicate or, inhibit the growth, proliferation and/or differentiation of embryonic stem cells associated with vascular anomalies, wherein the embryonic stem cells are characterized by (i) the expression of one or more stem cell biomarkers selected from the group consisting of Oct-4, SOX2, NANOG and PSTAT3, and (ii) the expression of one or more biomarkers associated with the Renin-Angiotensin System selected from the group consisting of Renin Receptor, a soluble form of Angiotensin Converting Enzyme (ACE), Angiotensin II Receptor 1, Angiotensin II Receptor 2 and a soluble form of the Renin Receptor, and wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

In yet another aspect of the present invention there is provided a method for determining the presence or absence of vascular anomalies in a subject, the method comprising:

(i) detecting and/or measuring the levels of embryonic stem cells present in a biological sample obtained from the subject using biomarker expression analysis;

(ii) comparing the levels of the embryonic stem cells obtained from the biological sample against the level from a control population;

wherein, an increased level in the embryonic stem cells obtained from the biological sample relative to the control population is diagnostic that the subject has, or is predisposed to developing, vascular anomalies.

In another aspect of the present invention there is provided a method for determining presence or absence of vascular anomalies in a subject, the method comprising:

(i) detecting and/or measuring the level of embryonic stem cells and/or components of the Renin-Angiotensin system in a biological sample obtained from the subject using biomarker expression analysis;

(ii) comparing the level of the embryonic stem cells and/or components of the Renin-Angiotensin system obtained from the biological sample against the level from a control population,

wherein, an increased level in the embryonic stem cells and/or components of the Renin-Angiotensin system obtained from the biological sample relative to the control population is diagnostic that the subject has, or is predisposed to developing, vascular anomalies, and

(iii) administering a prophylactic or therapeutic regime to the subject who has, or is predisposed to developing, vascular anomalies.

In another aspect of the present invention there is provided a pharmaceutical composition for use in a method for treatment of vascular anomalies, wherein the pharmaceutical composition comprises a therapeutic agent sufficient to selectively eradicate or, inhibit the growth, proliferation and/or differentiation of embryonic stem cells associated with vascular anomalies, and wherein the method comprises administering the therapeutic agent to a patient with vascular anomalies.

In another aspect of the present invention there is provided a kit or article of manufacture for use in the treatment of vascular anomalies, the kit comprising a therapeutic agent sufficient to selectively eradicate, or inhibit the growth, proliferation and/or differentiation of embryonic stem cells associated with vascular anomalies, together with instructions for how to administer a therapeutic dose to the subject.

### BRIEF DESCRIPTION OF THE FIGURES

FIG. 1 shows representative immunofluorescent immunohistochemical stained sections of pyogenic granuloma. The endothelial layer of the microvessels in PG expressed ERG (A&B, red) and CD34 (A, C-E, green). OCT4 (B, green) and ERG (B, red) were co-expressed by the endothelial layer (appearing as orange). SOX2 (C, red) was also expressed by the endothelial cells (green, arrowheads) and the interstitial cells (red, arrows). Phosphorylated STAT3 (D, red) was expressed by both the endothelial layer (arrowheads) and cells of the interstitium (arrows). NANOG (E, red) was also present in both the endothelial layer (arrowheads) and interstitial cells (arrows). Image magnification: 400×.

FIG. 2 shows copy number of mRNA transcripts in pyogenic granuloma (n=6) for NANOG, OCT4 and STAT3 expressed as a ratio over housekeeping gene GUSB. Error bars represent standard error of the mean.

FIG. 3 shows representative in situ hybridization stained formalin-fixed paraffin-embedded pyogenic granuloma (PG) sections demonstrating mRNA expression of OCT4 (A, pink, arrows), SOX2 (B, pink, arrows), STAT3 (C, pink, arrows) and NANOG (D, pink, arrows) in the endothelial lining of the microvessels of PG. Image magnification 400×.

FIG. 4 shows representative DAB IHC stained sections of pyogenic granuloma tissue demonstrating the expression of embryonic stem cell markers OCT4 (brown) and SOX2 (brown) on the endothelium, which also expresses Renin Receptor (brown) and Angiotensin Converting Enzyme (brown). The nuclei were counterstained with haematoxylin (blue). Image magnification: 400×.

FIG. 5 shows representative DAB IHC stained section of pyogenic granuloma demonstrating expression of the endothelial marker ERG (A, brown), which also expresses the embryonic stem cell markers OCT4 (B, brown), SOX2 (C, brown), pSTAT3 (D, brown) and NANOG (E, brown). Nuclei were counterstained with hematoxylin (blue). Image magnification: 400×.

FIG. 6 shows human seminoma tissue, as positive controls, displaying positive immunohistochemical immunofluorescent staining for OCT4 (A, green), SOX2 (B, red), pSTAT3 (C, red) and NANOG (D, red). Image magnification: 400×.

FIG. 7 shows human seminoma tissue, as positive controls, for in situ hybridization displaying mRNA expression for OCT4 (A, red), SOX2 (B, red), STAT3 (C, red) and NANOG (D, red). Image magnification: 400×.

FIG. 8 shows representative hematoxylin and eosin stained slides of IM (top) and SC (bottom) VM demonstrating thin-walled ectatic lesional vessels with variable smooth muscle cells. Original magnification: 100×.

FIG. 9 shows representative DAB IHC stained sections of subcutaneous (SC) (Right panels) and intramuscular (IM) venous malformations (Left panels) demonstrating the expression of PRR (top panels, brown). ACE was also expressed on the endothelium of both VM lesions (middle panels, brown). ATIIR1 was expressed in both IM and SC VM lesions (bottom panels, brown). Original magnification: 400×.

FIG. 10 shows representative DAB IHC stained section of SC (right panels) and IM (left panels) VM. ATIIR1 was expressed to a greater degree in the smaller lesional vessels (A&B, brown, thin arrows), compared with the adjacent larger lesional vessels (A&B, brown, thick arrows). Original magnification: 400×.

FIG. 11 Representative IF IHC-stained sections of IM (left panels) and SC (right panels) VM demonstrating the expression of PRR in IM and SC lesions (top panels, red). Endothelial cells were identified by the endothelial marker CD34 (top panels, green). ACE was also expressed on the endothelium of both IM and SC (middle panels, green) lesions. ATIIR1 was expressed in IM and SC (bottom panels, green) VM endothelium. The endothelial cells were identified using the endothelial marker ERG (middle and bottom panels, red). Cell nuclei were counterstained with 4′,6-diamidino-2-phenylindole (blue). Scale bars: 50 μm

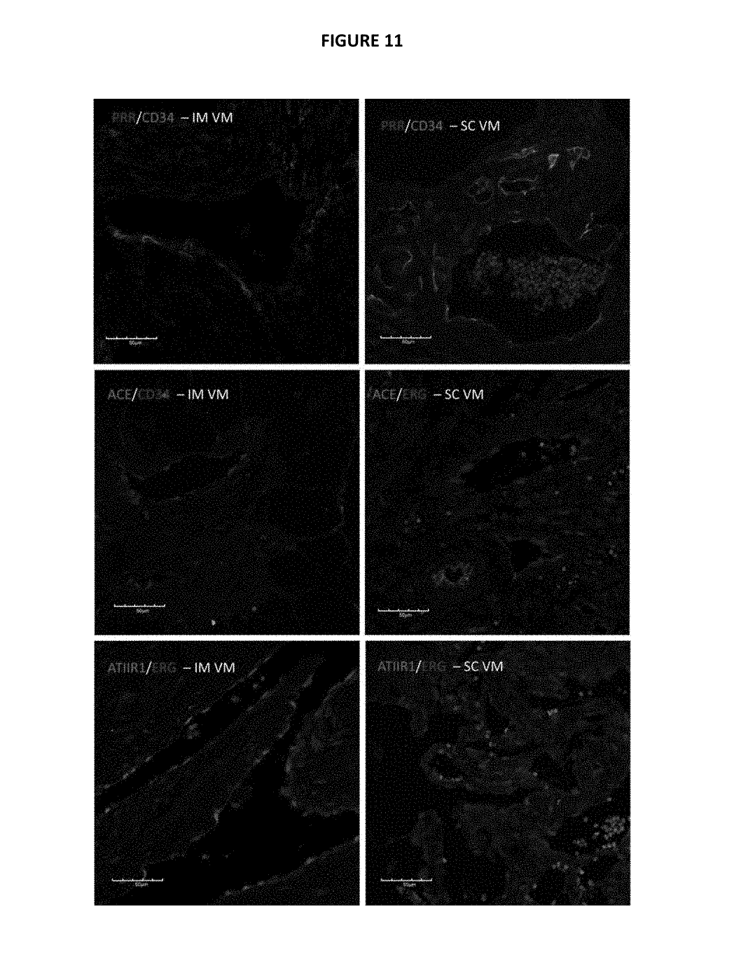

FIG. 12 shows Angiotensin Converting Enzyme (ACE), Angiotensin II Receptor 1 (ATIIR1) and Renin Receptor (ATP6AP2) mRNA levels in SC (n=3) and IM (n=3) VM tissues. Total mRNA was analyzed by NanoString nCounter Gene Expression assay with specific probes for ACE, ATIIR1, ATIIR2 and ATP6AP2 genes. Counts from ATIIR2 did not exceed those of the negative control. The data is presented as mean±SEM. Student's t-test was performed to evaluate significance (*p<0.05).

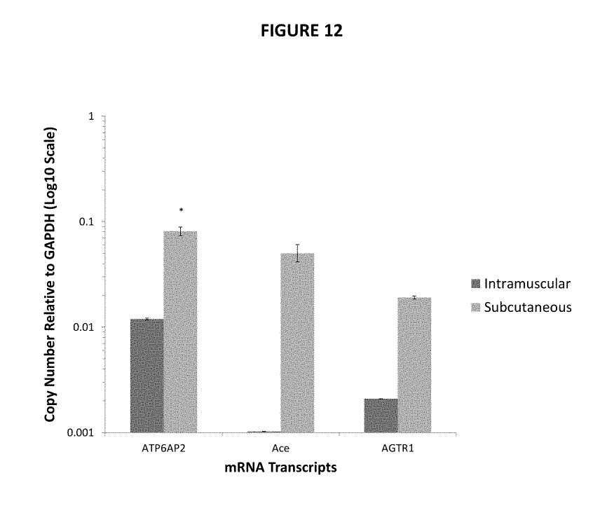

FIG. 13 shows representative DAB staining on venous malformations for the embryonic stem cell markers OCT4 (brown) and SOX2 (brown). Nuclei are counterstained with hematoxylin (blue). Original magnification: 400×.

FIG. 14 shows controls for DAB IHC staining: placenta stained positively for PRR (A, brown), kidney stained positively for ACE (B, brown), liver stained positively for ATIIR1 (C, brown), and kidney for ATIIR2 (D, brown). Specificity of the antibodies was demonstrated by omitting the primary antibody (E, brown). Original magnification: 400×.

FIG. 15 shows the main pathways associated with the RAS. ACE: Angiotensin Converting Enzyme; ACEIs: Angiotensin Converting Enzyme inhibitors; Cox2i: Cox2 inhibitors; β-blockers: Beta-Blockers; ATIIR2: Angiotensin II Receptor 2; ATIIR1: Angiotensin II Receptor 1; (Pro)-RR: Pro(Renin) Receptors [also called Renin Receptor (RR)]; Vit D: Vitamin D; XX: major blockades; ++: major promoting steps.

FIG. 16 shows the combined pathways associated with the RAS. ACE: Angiotensin Converting Enzyme; ACEI: Angiotensin Converting Enzyme Inhibitors; Cox2i: Cox2 inhibitors; n-blockers: Beta-Blockers; ATIIR2: Angiotensin II Receptor 2; ATIIR1: Angiotensin II Receptor 1; (Pro)-RR: Pro(Renin) Receptors [also called Renin Receptor (RR)]; Vit D: Vitamin D; XX: major blockades; X: minor blockades; ++: major promoting steps; +: minor blocking steps.

FIG. 17 shows representative H&E stained SCVM (A) and IMVM (B) sections demonstrating the characteristic ectatic venous channels. Representative sections of SCVM (C) and IMVM (D) showing minimal staining for D2-40 (C&D, brown). Nuclei were counterstained with hematoxylin (A-D, blue). Original magnifications: 400× (A&B) and 100× (C&D).

FIG. 18 shows DAB IHC-stained images demonstrating the expression of Nanog (A&B, red), pSTAT3 (C&D, brown), OCT4 (E&F, brown), SOX2 (G&H, brown), SALL4 (I&J, brown) and CD44 (K&L, brown) in SCVM (A, C, E, G, I & K) and IMVM (B, D, F, H, J & L). Endothelial staining of all six ESC markers was present on the endothelium within both SCVM and IMVM samples. Nanog, pSTAT3, SOX2 and CD44 were also expressed on cells (arrowheads) away from the endothelium in both SCVM and IMVM samples. Nuclei were counterstained with hematoxylin (blue). Original magnification: 400×.

FIG. 19 shows representative immunofluorescent immunohistochemical-stained sections of SCVM (A) and IMVM (B) samples, demonstrating the endothelium consisted of CD34⁺ (green)/ERG⁻ (red) (long arrows), ERG⁺(red)/CD34⁻ (green) endothelium (arrowheads) and CD34⁺ (red)/ERG⁺ (red) (short arrows) phenotypes. The CD34⁺ (green) endothelium expressed Nanog (red, arrows) in SCVM (C) and IMVM (D) lesions with cells away from the endothelium also expressing Nanog (red, arrowheads) within SCVM (C) and IMVM (D) lesions. The CD34⁺ (green) endothelium expressed pSTAT3 (red, arrows) in both SCVM (E) and IMVM (F) lesions. Cells away from the endothelium also expressed pSTAT3 (red, arrowheads) within SCVM (E) and IMVM (F) lesions. The ERG⁺ (red) endothelium also expressed OCT4 (green, arrows) in both SCVM (G) and IMVM (H) lesions. The CD34⁺ (green) endothelium expressed SOX2 (red, arrows) in SCVM (I) and IMVM (J) lesions. Cells away from the endothelium also expressed SOX2 (red, arrowheads) in SCVM (I) and IMVM (J) lesions. The ERG⁺ endothelium (red) expressed SALL4 (green, arrows) in SCVM (K) and IMVM (L) lesions. The ERG⁺ endothelium (red) expressed CD44 (green, arrows) in SCVM (M) and IMVM (N) lesions with cells away from the endothelium also expressing CD44 (green, arrowheads) in SCVM (M) and IMVM (N). Cells outside of the endothelium in both SCVM (O) and IMVM (P) co-expressed Nanog (O&P, red) and CD44 (O&P, green). Cell nuclei were counterstained with 4′,6′-diamidino-2-phenylindole (A-P, blue). Scale bars: 20 μm.

FIG. 20 shows Log₁₀ relative expression of OCT4, STAT3 and CD44 (A) and SOX2, Nanog and SALL4 (B) mRNA transcripts in three SCVM and three IMVM samples analyzed by NanoString (A) and RT-qPCR (B). Expression is depicted relative to the housekeeping gene GAPDH. OCT4 was detected in two SCVM and two IMVM samples (A). STAT3 and CD44 (A) and SOX2 and SALL4 (B) were detected in all samples. Nanog was detected in all three SCVM samples and two out of three IMVM samples (B).

FIG. 21 shows positive controls for Nanog (A, red), OCT4 (B, brown), SALL4 (C, brown), SOX2 (D, brown), pSTAT3 (E, brown), CD44 (F, brown). Negative controls for subcutaneous (G) and intramusacular (H) venous malformation. Nuclei were counterstained with hematoxylin (blue). Original magnification: (A-F) 400×; (G&H) 100×.

FIG. 22 shows negative control immunofluorescent immunohistochemical sections of subcutaneous (A) and intramusacular (B) venous malformation demonstrating minimal staining. Cell nuclei were counterstained with 4′,6′-diamidino-2-phenylindole (A-F, blue). Scale bars: 20 μm.

### SELECTED DEFINITIONS

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used herein, the words “comprises”, “comprising”, and similar words, are not to be interpreted in an exclusive or exhaustive sense. In other words, they are intended to mean “including, but not limited to”.

As used herein, the term “vascular anomalies” is intended to mean one or more vascular tumors or vascular malformations classified according to the classification system of the International Society for the Study of Vascular Anomalies.¹

As used herein, the term “antibodies” refer to molecules that contain an antigen binding site, e.g., immunoglobulins. Immunoglobulin molecules can be of any type (e.g., IgG, IgE, IgM, IgD, IgA and IgY), class (e.g., IgGI, IgG2, IgG3, IgG4, IgAI and IgA2) or subclass. Antibodies include, but are not limited to, monoclonal antibodies, polyclonal antibodies, multispecific antibodies, human antibodies, humanised antibodies, murine antibodies, camelised antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above.

As used herein, the term “effective amount” refers to the amount of a therapy that is sufficient to result in the prevention of the development, recurrence, or onset of a vascular anomaly and one or more symptoms thereof, to enhance or improve the prophylactic effect(s) of another therapy, reduce the severity, the duration of the vascular anomaly, ameliorate one or more symptoms of the vascular anomaly, prevent the advancement of the vascular anomaly, cause regression of the vascular anomaly, and/or enhance or improve the therapeutic effect(s) of another therapy.

As used herein, the terms “manage”, “managing”, and “management” in the context of the administration of a therapy to a subject refer to the beneficial effects that a subject derives from a therapy (e.g., a prophylactic or therapeutic agent) or a combination of therapies, while not resulting in a cure of a vascular anomaly. In certain examples, a subject is administered one or more therapies (e.g., one or more prophylactic or therapeutic agents) to “manage” the vascular anomaly so as to prevent the progression or worsening of the condition.

As used herein, the terms “prevent”, “preventing” and “prevention” in the context of the administration of a therapy to a subject refers to the prevention or inhibition of the recurrence, onset, and/or development of the vascular anomaly or a symptom thereof in a subject resulting from the administration of a therapy (e.g., a prophylactic or therapeutic agent), or a combination of therapies (e.g., a combination of prophylactic or therapeutic agents).

As used herein, the term “marker” or “biomarker” in the context of a tissue means any antigen, molecule or other chemical or biological entity that is specifically found in or on a tissue, or in circulation, that it is desired to be identified or identified in or on a particular tissue affected by a disease or disorder, for example a vascular anomaly. In specific examples, the marker is a cell surface antigen that is differentially or preferentially expressed by specific cell types. In specific examples, the marker is a nuclear antigen that is differentially or preferentially expressed by specific cell types. In specific examples the marker is an intracellular antigen that is differentially or preferentially expressed by specific cell types.

As used herein, the term “prophylactic agent” refers to any molecule, compound, and/or substance that is used for the purpose of preventing a vascular anomaly. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, proliferation based therapy, and small molecule drugs.

As used herein, the term “therapeutic agent” refers to any molecule, compound, and/or substance that is used for the purpose of treating and/or managing a disease or disorder, such as a vascular anomaly. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, proliferation-based therapy agents, hormonal agents, radioimmunotherapies, targeted agents, epigenetic therapies, differentiation therapies, biological agents, and small molecule drugs.

As used herein, the terms “therapies” and “therapy” can refer to any method(s), composition(s), and/or agent(s) that can be used in the prevention, treatment and/or management of vascular anomalies or one or more symptoms thereof.

As used herein, the terms “treat”, “treatment” and “treating” in the context of the administration of a therapy to a subject refer to the reduction or inhibition of the progression and/or duration of the vascular anomaly, the reduction or amelioration of the severity of the vascular anomaly, and/or the amelioration of one or more symptoms thereof resulting from the administration of one or more therapies.

The term “sample” or “biological sample” as used herein means any sample taken or derived from a subject. Such a sample may be obtained from a subject, or may be obtained from biological materials intended to be provided to the subject. For example, a sample may be obtained from blood being assessed, for example, to investigate a vascular anomaly in a subject. Included are samples taken or derived from any subjects such as from normal healthy subjects and/or healthy subjects for whom it is useful to understand their vascular anomaly status. Preferred samples are biological fluid samples. The term “biological fluid sample” as used herein refers to a sample of bodily fluid obtained for the purpose of, for example, diagnosis, prognosis, classification or evaluation of a subject of interest, such as a patient. The sample may be any sample known in the art in which embryonic stem cells may be detected. Included are any body fluids such as a whole blood sample, plasma, serum, ovarian follicular fluid sample, seminal fluid sample, cerebrospinal fluid, saliva, sputum, urine, pleural effusions, interstitial fluid, synovial fluid, lymph, tears, for example, although whole blood sample, plasma and serum are particularly suited for use in this invention. In addition, one of skill in the art would realise that certain body fluid samples would be more readily analysed following a fractionation or purification procedure, for example, separation of whole blood into serum or plasma components.

The term “purified” as used herein does not require absolute purity. Purified refers in one embodiment to at least 90%, or 95%, or 98%, or 99% homogeneity of, to provide an example, of a polypeptide or antibody in a sample.

The term “subject” as used herein is preferably a mammal and includes human, and non-human mammals such as cats, dogs, horses, cows, sheep, deer, mice, rats, primates (including gorillas, rhesus monkeys and chimpanzees), possums and other domestic farm or zoo animals. Thus, the assays, methods and kits described herein have application to both human and non-human animals, in particular, and without limitation, humans, primates, farm animals including cattle, sheep, goats, pigs, deer, alpacas, llamas, buffalo, companion and/or pure bred animals including cats, dogs and horses. Preferred subjects are humans, and most preferably “patients” who as used herein refer to living humans who may receive or are receiving medical care or assessment for a disease or condition. Further, while a subject is preferably a living organism, the invention described herein may be used in post-mortem analysis as well.

The term “ELISA” as used herein means an enzyme linked immunosorbent assay, a type of competitive binding assay comprising antibodies and a detectable label used to quantitate the amount of an analyte in a sample.

The term “capture antibody” as used herein means an antibody that is typically immobilized on a solid support such as a plate, bead or tube, and which antibody binds to and captures analyte(s) of interest, for example membrane-bound markers associated with an embryonic stem cell population.

The term “detection antibody” as used herein means an antibody comprising a detectable label that binds to an analyte(s) of interest. The label may be detected using routine detection means for a quantitative, semi-quantitative or qualitative measure of the analyte(s) of interest, for example membrane-bound markers associated with an embryonic stem cell population.

As used herein, the term “relating to the presence or amount” of an analyte reflects that assay signals are typically related to the presence or amount of an analyte through the use of a standard curve calculated using known concentrations of the analyte of interest. As the term is used herein, an assay is “configured to detect” an analyte if an assay can generate a detectable signal indicative of the presence or amount of a physiologically relevant concentration of the analyte. Typically, an analyte is measured in a sample.

A level “higher” or “lower” than a control, or a “change” or “deviation” from a control (level) in one embodiment is statistically significant. A higher level, lower level, deviation from, or change from a control level or mean or historical control level can be considered to exist if the level differs from the control level by about 5% or more, by about 10% or more, by about 20% or more, or by about 50% or more compared to the control level. Statistically significant may alternatively be calculated as P≦0.05. Higher levels, lower levels, deviation, and changes can also be determined by recourse to assay reference limits or reference intervals. These can be calculated from intuitive assessment or non-parametric methods. Overall, these methods may calculate the 0.025, and 0.975 fractiles as 0.025*(n+1) and 0.975 (n+1). Such methods are well known in the art. Presence of a marker absent in a control may be seen as a higher level, deviation or change. Absence of a marker present in a control may be seen as a lower level, deviation or change.

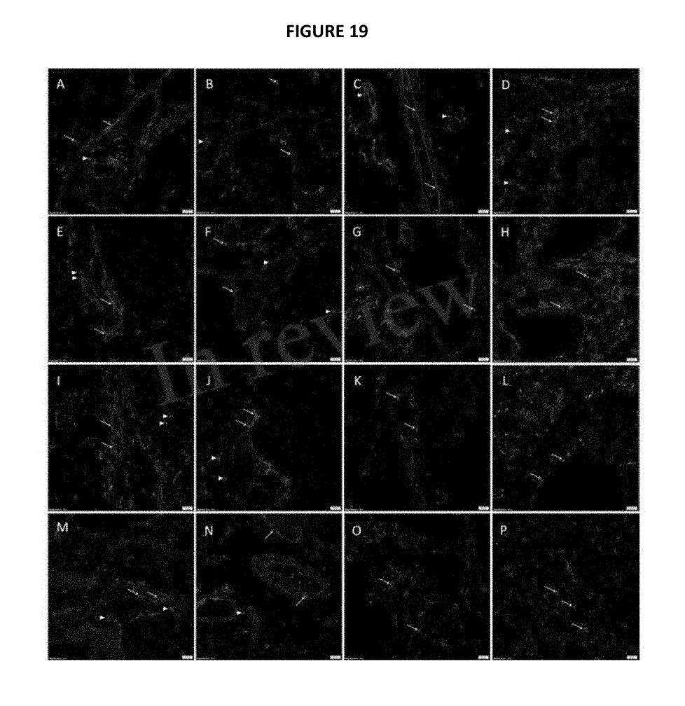

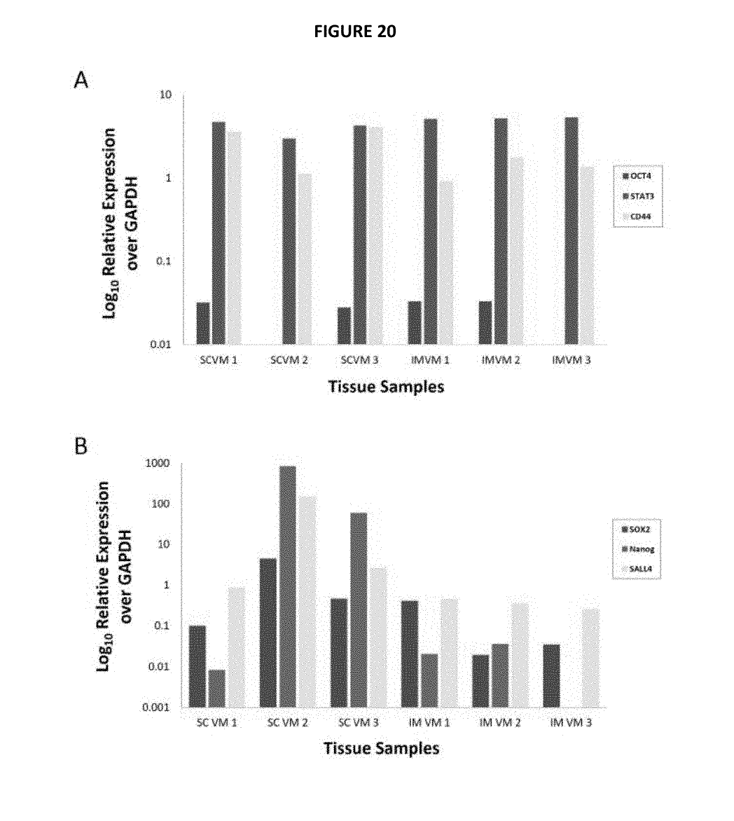

As used herein, the term “Renin-Angiotensin System (RAS)” or “Renin-Angiotensin-Aldosterone System (RAAS)” is a hormone system that regulates blood pressure and fluid balance. The wider pathway associated with RAS also includes the Pro/Renin Receptor System (PRRS) and the associated bypass pathways. By way of example, refer to FIGS. 19 and 20. There are a number of known drugs which target the RAS including PRRS, as described in more detail below.

### DETAILED DESCRIPTION

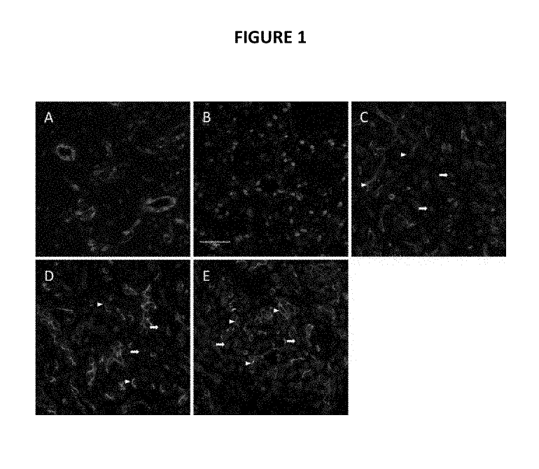

The present invention is predicated on the surprising and unexpected discovery that discrete populations of embryonic stem cells are associated with vascular anomalies such as, for example, venous malformation, pyogenic granuloma and infantile hemangioma. The embryonic stem cell populations associated with these conditions may be characterised by unique biomarker expression profiles that allow for the specific identification and diagnosis of vascular anomalies. Refer to, for example, FIGS. 1-4 in respect of pyogenic granuloma as an example of vascular anomalies.

Expression of embryonic-like stem cell populations in tissues affected by vascular anomalies may be crucial in the development and pathology of vascular anomalies.

Examples 1-3 describe expression of embryonic stem cell markers, OCT4, SOX2, STAT3 and NANOG in PG samples from six patients, by immunohistochemical (IHC) staining, NanoString analysis and in-situ hybridization (ISH).

IHC staining demonstrated the expression of pSTAT3, OCT4, SOX2 and NANOG by the endothelium of the microvessels in PG whilst pSTAT3, SOX2 and NANOG were also expressed by cells in the interstitium, outside of the microvessels. NanoString and ISH analysis showed mRNA expression for STAT3, OCT4 and NANOG in PG.

The expression of the ESC markers, OCT4, SOX2, pSTAT3 and NANOG, suggests the endothelium of PG displays a primitive phenotype. Cells in the interstitium expressing pSTAT3, SOX2 and NANOG may represent a more downstream derivative of the primitive endothelium, or a separate population. The primitive nature of the endothelium and cells in the interstitium reveals novel insights into the biology of PG.

Similarly, Examples 4-6 investigate the expression of components of the Renin-Angiotensin System (RAS), (pro)renin receptor (PRR), angiotensin converting enzyme (ACE), angiotensin II receptor 1 (ATIIR1) and angiotensin II receptor 2 (AIITR2) in subcutaneous (SC) and intramuscular (IM) venous malformation. SC (n=7) and IM (n=7) VM were analyzed for the expression of PRR, ACE, ATIIR1, and ATIIR2 using immunohistochemical (IHC) staining, NanoString gene expression analysis and Western blotting (WB).

Note, Renin Receptor is also known in the art by the term ATP6AP2.

IHC staining showed expression of PRR, ACE and ATIIR1 but not ATIIR2 in the endothelium of SC and IM VM. These results were confirmed by NanoString analysis. WB demonstrated the presence of PRR and ATIIR1 but not ACE and ATIIR2.

The presence of PRR, ACE and ATIIR1, at both the transcriptional and translational levels in both SC and IM VM suggests a putative role for the RAS in the biology VM. This novel finding may lead to a mechanism-based therapy for VM.

Accordingly, in one aspect of the present invention there is provided a method for preventing, treating, or managing vascular anomalies in a patient in need thereof, the method comprising administering a therapeutic agent to the patient in an amount sufficient to selectively eliminate or inhibit the growth, proliferation and/or differentiation of embryonic stem cells associated with vascular anomalies, wherein the embryonic stem cells are characterized by the expression of one or more embryonic stem cell biomarkers.

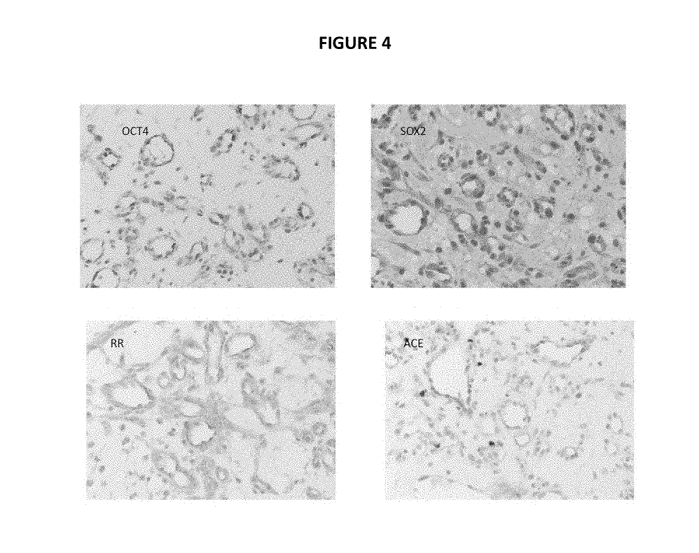

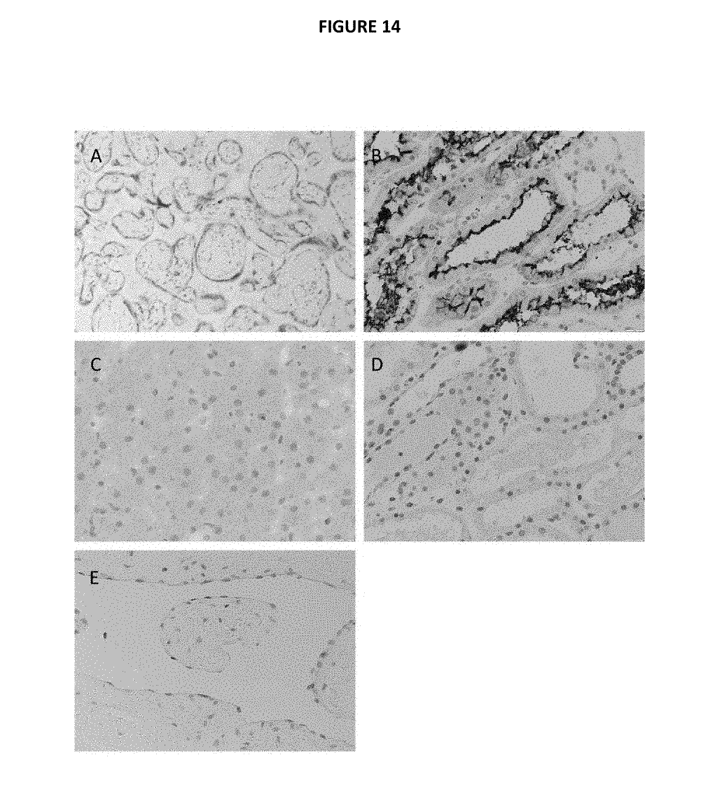

Further, it has also been revealed by the Applicants that these embryonic-like stem cell populations express key components of the Renin-Agiotensin System (RAS), including the Renin Receptor (RR) and Angiotensin Converting Enzyme (ACE). In reference to FIGS. 4 and 14, Applicants demonstrate co-expression of Renin Receptor and soluble form of Angiotensin Converting enzyme (ACE) by the embryonic stem cell populations associated with pyogenic granuloma and venous malformation, as examples of vascular anomalies. These embryonic stem cell populations are characterised by, for example, the expression of OCT4, SOX2, PSTAT3 and NANOG. Further, it is possible that these embryonic stem cells also express Angiotensin II Receptor 1 (ATIIR1), Angiotensin II Receptor 2 (ATIIR2), as well as soluble forms of the Renin Receptor (sRR). Accordingly, the expression of the components of RAS by these embryonic stem cell populations provides a novel and unique therapeutic approach by targeting the embryonic stem cells associated with vascular anomalies from the extensive array of drugs that target RAS such as, Angiotensin-Converting Enzyme Inhibitors (ACEis), Angiotensin Receptor Blockers (ARBs), Direct Renin Inhibitors (DRIs), Beta-Blockers, Cyclo-oxygenase 2 Inhibitors, Chymase Inhibitors, Inhibitors of Cathepsin B, Cathepsin D and Cathepsin G, Calcium Supplements, Vitamin D and Calcium Channel Blockers.

In addition, the present invention also contemplates indirect inhibitors of the RAS (e.g., Calcium Channel Blockers).

Accordingly, in another aspect of the present invention there is provided a method for preventing, treating, or managing vascular anomalies in a patient in need thereof, the method comprising administering a therapeutic agent to the patient in an amount sufficient to selectively eliminate or inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System.

In yet another aspect of the present invention there is provided a method for preventing, treating, or managing vascular anomalies in a patient in need thereof, the method comprising administering a therapeutic agent to the patient in an amount sufficient to selectively inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System, and wherein vascular anomalies is selected from the group consisting of pyogenic granuloma, venous malformation and infantile hemangioma, lymphatic malformation, capillary malformation, arterial malformation, arterio-venous malformation, lymphatico-venous malformation, capillary-venous malformation, capillary-lymphatico-venous malformation, kaposiform hemangioendotheloima, tufted angioma, hepatic hemangioendothelioma and verrucous hemangioma.

In one example according to the present invention, vascular anomalies is venous malformation or pyogenic granuloma.

In another aspect of the present invention there is provided a method for preventing, treating, or managing vascular anomalies in a patient in need thereof, the method comprising administering a therapeutic agent to the patient in an amount sufficient to selectively inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System, and wherein vascular anomalies is pyogenic granuloma.

In yet another aspect of the present invention there is provided a method for preventing, treating, or managing vascular anomalies in a patient in need thereof, the method comprising administering a therapeutic agent to the patient in an amount sufficient to selectively inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System, and wherein vascular anomalies is venous malformation.

In one example, the one or more embryonic stem cell markers is selected from the group consisting of Cripto, ABCG2, Alkaline Phosphatase/ALPL, CD9, FGF-4, GDF-3, Integrin alpha 6/CD49f, Integrin beta 1/CD29, Nanog, Oct-3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1. In another example, the one or more embryonic stem cell biomarkers consists in OCT4, SOX2, NANOG and pSTAT3. In yet another example, the one or more biomarkers associated with the RAS is selected from the group consisting of Renin Receptor, Angiotensin II Receptors, a soluble form of the Renin Receptor and a soluble form of Angiotensin Converting Enzyme.

In another aspect of the present invention there is provided a method for preventing, treating, or managing vascular anomalies in a patient in need thereof, the method comprising administering a therapeutic agent to the patient in an amount sufficient to selectively eliminate, or inhibit the growth, proliferation and/or differentiation of embryonic stem cells associated with vascular anomalies, wherein the embryonic stem cells are characterized by (i) the expression of one or more stem cell biomarker selected from the group consisting of Cripto, ABCG2, Alkaline Phosphatase/ALPL, CD9, FGF-4, GDF-3, Integrin alpha 6/CD49f, Integrin beta 1/CD29, NANOG, OCT3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, Oct-4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1, and (ii) the expression of one or more biomarkers associated with the Renin-Angiotensin System.

In yet another aspect of the present invention there is provided a method for preventing, treating, or managing vascular anomalies in a patient in need thereof, the method comprising administering a therapeutic agent to the patient in an amount sufficient to selectively eliminate, or inhibit the growth, proliferation and/or differentiation of embryonic stem cells associated with vascular anomalies, wherein the embryonic stem cells are characterized by (i) the expression of one or more stem cell marker selected from the group consisting of OCT4, SOX2, NANOG and PSTAT3, and (ii) the expression of one or more biomarkers associated with the Renin-Angiotensin System selected from the group consisting of Renin Receptor, Angiotensin II Receptor 1, Angiotensin II Receptor 2, a soluble form of the Renin Receptor and a soluble form of Angiotensin Converting Enzyme.

In yet a further aspect of the present invention there is provided a method for preventing, treating, or managing vascular anomalies in a patient in need thereof, the method comprising administering a therapeutic agent to the patient in an amount sufficient to selectively eradicate, or inhibit the growth, proliferation and/or differentiation of embryonic stem cells associated with vascular anomalies, wherein the embryonic stem cells are characterized by (i) the expression of one or more stem cell biomarker selected from the group consisting of OCT4, SOX2, NANOG and PSTAT3, and (ii) the expression of Renin-Angiotensin System marker selected from Renin Receptor, soluble form of Angiotensin Converting Enzyme, Angiotensin II Receptor 1, Angiotensin II Receptor 2 and/or a secreted form of Renin Receptor, and wherein the therapeutic agent is selected from the group consisting of Direct Renin Inhibitors (DRIs), ACEis, ARBs, Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

In one example, the embryonic stem cells are characterized by the expression of SOX2, PSTAT3 and NANOG. These cells are said to have a marker expression profile: SOX2⁺ PSTAT3⁺NANOG⁺.

In one example, the embryonic stem cells are characterized by the expression of SOX2, OCT4, PSTAT3 and NANOG. These cells are said to have a marker expression profile: SOX2⁺OCT4⁺PSTAT3⁺NANOG⁺.

In a related example, the embryonic stem cells are embryonic stem cells associated with vascular anomalies and are characterized by the marker expression profile SOX2⁺OCT4⁺NANOG⁺PSTAT3⁺. In a further example, the embryonic stem cells associated with vascular anomalies are characterized by the marker expression profile SOX2⁺OCT4⁺NANOG⁺PSTAT3⁺CD34⁺. In yet a further example, the embryonic stem cells associated with vascular anomalies are characterized by the marker expression profile SOX2⁺PSTAT3⁺NANOG⁺CD34⁻.

The embryonic stem cells may co-express with other embryonic stem cell markers, lymphatic cell markers, or any combination thereof.

The present invention provides compositions and methods related to identifying and targeting the growth and proliferation of embryonic stem cells as the cause of vascular anomalies. By specifically targeting these embryonic stem cells, it is assumed that the lesion potential is significantly diminished, thereby leading to enhanced therapeutic outcomes.

The embryonic stem cells may be associated with a variety of vascular anomalies, including but not limited to, pyogenic granuloma, venous malformation, infantile hemangioma, lymphatic malformation, capillary malformation, arterial malformation, arterio-venous malformation, lymphatico-venous malformation, capillary-venous malformation, capillary-lymphatico-venous malformation, kaposiform hemangioendotheloima, tufted angioma, hepatic hemangioendothelioma, verrucous hemangioma.

The present invention provides methods for preventing, treating, and/or managing vascular anomalies, the method comprising administering to a subject in need thereof a course of therapy that stabilises, reduces, or eradicate the embryonic stem cell population. In certain examples, the stabilization, reduction, or elimination of the embryonic stem cell population is achieved by administering a therapy that targets the growth and proliferation of the stem cells.

Surprisingly, Applicants demonstrate that the embryonic stem cell populations identified in the methods according to the present invention co-express components of RAS in multiple different vascular anomalies. By way of illustration only, the Applicants demonstrate co-expression of the RR and a soluble form of ACE with embryonic stem cells shown to be associated with pyogenic granuloma (FIG. 4).

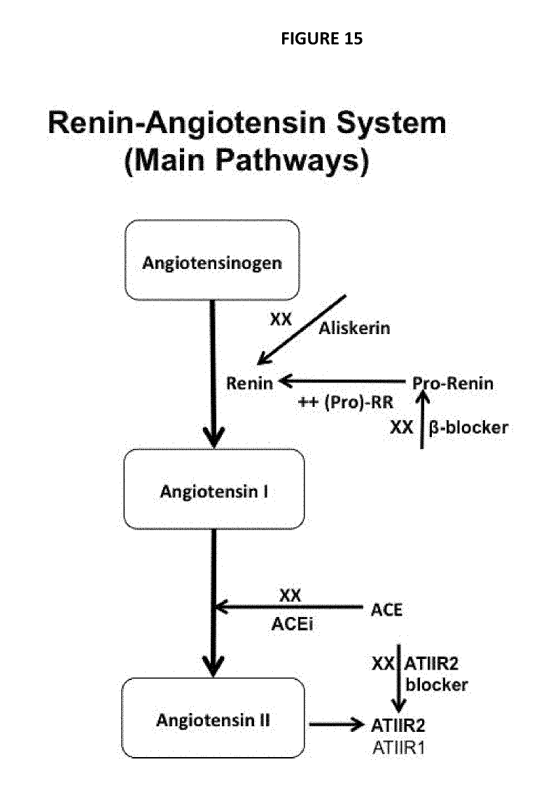

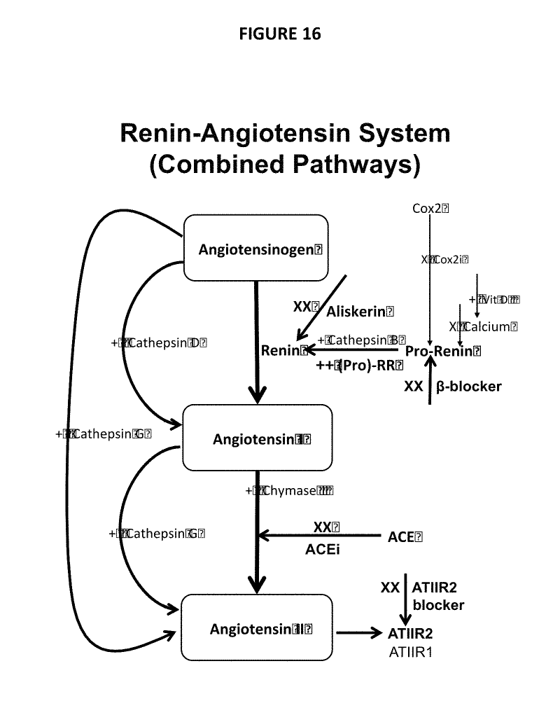

Accordingly, therapy that targets the growth and proliferation of embryonic stem cell populations comprises administering a therapeutic agent that selectively targets components of the RAS and/or Pro/Renin Receptor Systems (PRRS) expressed by the stem cells. FIGS. 15 and 16 show the types of inhibitors/drugs that target these systems, useful in accordance with the compositions and methods according to the present invention.

Examples of known therapeutics that target the Renin-Angiotensin System include, but are not limited to, ACEIs, ARBs, DRIs, Beta-Blockers, Cyclo-oxygenase 2 Inhibitors, Inhibitors of Cathepsin B, Cathepsin D and Cathepsin G, Calcium Channel Blockers, Calcium Supplements and Vitamin D.

Examples of ACEIs include, but are not limited to, Benazepril (Lotesin), Captopril (Capoten), Cilazipril, Enalapril (Vasotec, Renitec), Fosinopril (Monopril), Lisinopril (Lisodur, Lopril, Novatec, Prinivil, Zestril), Moexipril, Perindopril (Coversay, Aceon), Quinapril (Accupril), Ramipril (Altace, Tritace, Ramace, Ramiwin), Trandolapril, Delapril, Zofenopril and Imidapril.

Examples of ARBs include, but are not limited to, Losartan, Irbesartan, Candesartan, Eprosartan, Olmesartan, Telmisartan, PD123319 and Valsartan.

Examples of Beta-Blockers include, but are not limited to, Acebutolol (Sectral), Atenolol (Tenormin), Betaxolol (Betoptic), Bisoprolol (Cardicor, Emcor, Zebeta), Carteolol (Teoptic), Carvedilol (Coreg, Eucardic), Celiprolol (Celectol), Labetalol (Trandate), Levobunolol (Betagan), Metipranolol (Metipranolol Minims), Metoprolol (Betaloc, Lopresor, Lopressor, Toprol XL), Nadolol (Corgard), Nebivolol (Bystolic, Nebilet), Oxprenolol (Trasicor), Pindolol (Visken), Propranolol (Inderal LA), Sotalol (Beta-Cardone, Sotacor), and Timolol (Betim, Nyogel, Timoptol).

Examples of Cyclo-oxygenase 2 Inhibitors include, but are not limited to, Celecoxib, Nepafenac, Ibuprofen (Dolgesic), Indomethacin, Sulindac, Xanthohumol, Meclofenamate Sodium, Meloxicam, Rofecoxib, Bromfenac Sodium, Ibuprofen Lysine, Ketorolac (Ketorolac tromethamine), Diclofenac Sodium, Etodolac, Ketoprofen, Naproxen Sodium, Piroxicam, Acemetacin, Phenacetin, Tolfenamic Acid, Nimesulide, Flunixin Meglumin, Aspirin, Bufexamac, Niflumic acid, Licofelone, Oxaprozin, Lornoxicam, Lumiracoxib, Zaltoprofen, Ampiroxicam, Valdecoxib, Nabumetone, Mefenamic Acid, Carprofen, Amfenac Sodium monohydrate, Curcumin, Asaraldehyde and Suprofen.

Examples of Chymase Inhibitors include, but are not limited to, TY-51469 (2-[4-(5-fluoro-3-methylbenzo[b]thiophen-2-yl)sulfonamido-3-methanesulfonyl-phenyl]thiazole-4-carboxylic acid), Eglin C, CI, SUN13834, Chymostatin, TJK002 a benzimidazole inhibitor, ONO-WH-236, Amblyomma americanum tick serine protease inhibitor 6 (AamS6), N-tosyl-L-phenylalanyl chloromethyl ketone (TPCK), Alpha-aminoalkylphosphonate diaryl esters, Serine protease inhibitor A3 (serpinA3), Squamous cell carcinoma antigen (SCCA-2), Bortezomib (Velcade), R05066852 and 17beta-estradiol.

Examples of Cathepsin B Inhibitors include, but are not limited to, Cystatin B, Curcumin, Cystatin C, Cysteine peptidase inhibitor E64, [Pt(dmba)(aza-N1)(dmso)] complex 1 (a potential anti-tumoral drug with lower IC50 than cisplatin in several tumoral cell lines), 2,3,7,8-Tetrachlorodibenzo-p-dioxin (TCDD), CA-074Me, Lipidated CtsB inhibitor incorporated into the envelope of a liposomal nanocarrier (LNC-NS-629), Proanthocyanidin (PA) and ahpatinin Ac (1) and ahpatinin Pr (2).

Examples of Cathepsin D Inhibitors include, but are not limited to, non-peptidic acylguanidine inhibitors of Cathepsin D, Pepstatin A, Bm-Aspin, SIPI, Via, RNAi-Rab27A and Solanum lycopersicum aspartic protease inhibitor (SLAPI).

Examples of Cathepsin G Inhibitors include, but are not limited to, WFDC12, Phenylmethylsulfonyl fluoride (PMSF), Ecotin, SerpinBl, SerpinA3, CeEI, or Caesalpinia echinata elastase inhibitor, SLPI (secretory leukocyte protease inhibitor), Alpha1-Antitrypsin (AAT), Bauhinia bauhinoides cruzipain inhibitor, Alpha-Aminoalkylphosphonate diaryl esters, Greglin, [2-[3-[[(1-benzoyl-4-piperidinyl)methylamino]carbonyl]-2-naphthalenyl]-1-(1-naphthalenyl)-2-oxoethyl]-phosphonic acid (KPA), Lympho-Epithelial Kazal-Type-related Inhibitor (LEKTI), Trappin-2 A62L, SV-66, SCGI, Bortezomib, Human monocyte/neutrophil elastase inhibitor (MNEI), a 42-kDa serpin protein and Anti-leukoproteinase (ALP).

Examples of Calcium Channel Blockers include, but are not limited to, Dihydropyridine Calcium Channel Blockers, Phenylalkylamine Calcium Channel Blockers, Benzothiazepine Calcium Channel Blockers, Non-Selective Calcium Channel Blockers, as well as “Other” Calcium Channel blockers.

Examples of Dihydropyridine Calcium Channel Blockers include, but are not limited to, Amlodipine (Norvasc), Aranidipine (Sapresta), Azelnidipine (Calblock), Barnidipine (HypoCa), Benidipine (Coniel), Cilnidipine (Atelec, Cinalong, Siscard) Not available in US, Clevidipine (Cleviprex), Isradipine (DynaCirc, Prescal), Efonidipine (Landel), Felodipine (Plendil), Lacidipine (Motens, Lacipil), Lercanidipine (Zanidip), Manidipine (Calslot, Madipine), Nicardipine (Cardene, Carden SR), Nifedipine (Procardia, Adalat), Nilvadipine (Nivadil), Nimodipine (Nimotop), Nisoldipine (Baymycard, Sular, Syscor), Nitrendipine (Cardif, Nitrepin, Baylotensin), Pranidipine (Acalas).

Examples of Phenylalkylamine Calcium Channel Blockers include, but are not limited to, Verapamil (Calan, Isoptin), Gallopamil and Fendiline.

Examples of Benzothiazepine Calcium Channel Blockers include, but are not limited to, Diltiazem (Cardizem) and Fendiline.

Examples of Non-Selective Calcium Channel Blockers include, but are not limited to, Mibefradil, Bepridil, Flunarizine, Fluspirilene and Fendiline.

Examples of other Calcium Channel Blockers include, but are not limited to, Gabapentin, Pregabalin and Ziconotide.

An example of DRIs includes, but is not limited to, Aliskiren.

In certain examples, the embryonic stem cells may be partially differentiated and committed toward a specific cell lineage associated with a particular vascular anomaly.

The present invention also contemplates identification of embryonic stem cells as a means of diagnosing vascular anomalies, by profiling expression of certain markers known to be associated with the embryonic stem cells.

Accordingly, in another aspect of the present invention there is provided a method for determining presence or absence of vascular anomalies in a subject, the method comprising:

(i) detecting and/or measuring the levels of embryonic stem cells present in a biological sample obtained from the subject using biomarker expression analysis;

(ii) comparing the levels of the embryonic stem cells obtained from the biological sample against the embryonic stem cell level from a control population;

wherein, an increased level in the embryonic stem cells obtained from the biological sample relative to the control population is diagnostic that the subject has, or is predisposed to developing, vascular anomalies.

The present invention further contemplates identification of embryonic stem cells as a means of diagnosing vascular anomalies, by profiling expression markers of the Renin Angiotensin system known to be associated with the embryonic stem cells.

Accordingly, in another aspect of the present invention there is provided a method for determining presence or absence of vascular anomalies in a subject, the method comprising:

(i) detecting and/or measuring the level of embryonic stem cells and/or components of the Renin-Angiotensin system in a biological sample obtained from the subject using biomarker expression analysis;

(ii) comparing the level of the embryonic stem cells and/or components of the Renin-Angiotensin system obtained from the biological sample against the stem cell level from a control population,

wherein, an increased level in the embryonic stem cells obtained from the biological sample relative to the control population is diagnostic that the subject has, or is predisposed to developing, vascular anomalies, and

(iii) administering a prophylactic or therapeutic regime to the subject who has, or is predisposed to developing, vascular anomalies.

The present invention also contemplates pharmaceutical compositions comprising a therapeutic active or drug useful in the treatment of vascular anomalies.

Accordingly, in another aspect of the present invention there is provided a pharmaceutical composition for use in a method for treatment of vascular anomalies, wherein the pharmaceutical composition comprises a therapeutic agent(s) sufficient to selectively eradicate or, inhibit the growth, proliferation and/or differentiation of embryonic stem cells associated with vascular anomalies, and wherein the method comprises administering the therapeutic agent to a patient with vascular anomalies.

In certain examples of the pharmaceutical compositions according to the present invention, the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

The present invention also contemplates kits and articles of manufacture comprising a therapeutic active or drug useful in the treatment of vascular anomalies.

Accordingly, in another aspect of the present invention there is provided a kit for use in the treatment of vascular anomalies, the kit comprising a therapeutic agent sufficient to selectively eradicate, or inhibit the growth, proliferation and/or differentiation of embryonic stem cells associated with vascular anomalies, together with instructions on how to administer a therapeutic dose to the subject.

In certain examples of the kits according to the present invention, the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

Further detail with respect to the present invention is presented under the following sub-headings.

### Renin-Angiotensin System

The Renin-Angiotensin System (RAS) is traditionally known to preserve fluid volume during periods of restricted dietary salt and also prevents ischaemia during acute volume loss. The main effector peptide of the RAS is angiotensin II (ATII). It induces vasoconstriction and sympathetic activation, raises aldosterone levels, and promotes renal salt and water retention via the angiotensin II receptor 1 (ATIIR1). Over the last few decades, the RAS has been a drug target of particular interest because of its involvement in cardiovascular disease (CVD) and renovascular disease. The CVD and renovascular disease can be understood as a continuum of risk factors, target organ damage, events, and mortality. Risk factors (such as hypertension, dyslipidemia, diabetes, and smoking) led to the development of target organ damage including atherosclerosis, left ventricular hypertrophy (LVH), and renal impairment. Target organ damage progressively worsens, leading ultimately to myocardial infarction (MI), heart failure (HF), end-stage renal disease (ESRD), stroke, or death.

ATII, the main effector peptide of the RAS, plays an active role during all stages of this continuum. The first step in the RAS cascade is the formation of angiotensin I (ATI) from the precursor angiotensinogen under the action of renin; early evidence for the importance of RAS in CVD came from the consistent finding that renin activity is predictive of the risk of cardiovascular (CV) events. ATI is then converted to ATII, the principal effector peptide of the RAS, by angiotensin-converting enzyme (ACE). In addition, ATII can be produced in tissues by enzymes such as chymase. This locally produced ATII is believed to mediate paracrine and autocrine functions. ATII acts via ATIIR1 and ATIIR2. Activation of ATIIR1 results in vasoconstriction, aldosterone and vasopressin secretion, sodium retention, and decreased renal perfusion. Hence, these receptors mediate the deleterious effects of ATII, including elevated blood pressure (BP) and cardiac and vascular remodeling. The effects of the ATII receptors have been less clearly defined because of the limited expression of these receptors in adults, because of their unconventional signalling pathways, and because many ATII-mediated actions are masked by opposing ATI-mediated effects. However, it is now recognised that ATIIR2 generally opposes the actions of ATIIR1, mediating various antiproliferative and anti-inflammatory effects and promoting tissue differentiation and regeneration and apoptosis.

Additional components of the RAS have been identified in the last decade, including bioactive angiotensin peptides, such as angiotensin III, angiotensin IV, and angiotensin-(1-7), the effects of which have not yet been fully elucidated for the CV and renal system.

The discovery of the renin receptor has shed further light on the biology of the RAS. Renin, simply considered until recently as the rate-limiting enzyme of RAS activation, has also turned out to be the ligand for a protein known as the renin/prorenin receptor that binds renin and prorenin about equally, regardless of their biologic activities. Prorenin, which represents 70% to 90% of total circulating renin, when bound to the receptor induces an increase in the catalytic efficiency of angiotensinogen conversion to ATI, which contributes to local production of ATII and its systemic levels, as well as binding of renin/prorenin to the renin/prorenin receptor, exerting physiologic effects that are independent of ATII, including activation of intracellular signal pathways, enhanced synthesis of DNA, and stimulation of the release of plasminogen activator inhibitor 1, collagen 1, fibronectin, and transforming growth factor β-1.6

There are a number of known drugs which target the RAS. The two major classes of drugs that target the RAS are the angiotensin-converting enzyme (ACE) inhibitors and the selective ATI receptor blockers (ARBs). Although both of these drug classes target ATII, the differences in their mechanisms of action have implications for their effects on other pathways and receptors that may have therapeutic implications. Both ACEIs and ARBs are effective antihypertensive agents that have been shown to reduce the risk of cardiovascular and renal events.

Direct inhibition of renin, the most proximal aspect of the RAS, became clinically feasible from 2007 with the introduction of Aliskiren. This latter drug has been shown to be efficacious for the management of hypertension. Combined therapy of direct renin-inhibitors with ACEIs or ARBs has been tested in some clinical situations such as congestive heart failure (HF) and proteinuria with diverse results.

RAS drugs include, but are not limited to, Angiotensin-Converting Enzyme Inhibitors (ACEIs), Angiotensin Receptor Blockers (ARBs), Direct Renin Inhibitors (DRIs), Beta-Blockers, Cyclo-oxygenase 2 Inhibitors, Chymase Inhibitors, Cathepsin Inhibitors including Cathepsin B Inhibitors, Cathepsin D Inhibitors and Cathepsin G Inhibitors, Calcium Channel Blockers, Calcium Supplements and Vitamin D, as described above.

### Articles of Manufacture

The present invention also encompasses a finished packaged and labeled pharmaceutical product(s). This article of manufacture includes the appropriate unit dosage form in an appropriate vessel or container such as a glass vial or other container that is hermetically sealed. The pharmaceutical product may contain, for example, a prophylactic or therapeutic agent in a unit dosage form in a first container, and in a second container, sterile water for injection. Alternatively, the unit dosage form may be a solid suitable for oral, transdermal, intranasal, or topical delivery.

In a specific example, the unit dosage form is suitable for intravenous, intramuscular, intranasal, oral, topical or subcutaneous delivery. Thus, the invention encompasses solutions, preferably sterile, suitable for each delivery route.

In some examples, the pharmaceutical product is a prophylactic and/or therapeutic agent disclosed herein. In some examples, the pharmaceutical product is a composition comprising a prophylactic and/or therapeutic agent and a pharmaceutically acceptable carrier or excipient. In a specific example, the pharmaceutical composition is in a form for an appropriate route of administration. Such routes include, without limitation, oral, topical, parenteral, sublingual, rectal, vaginal, ocular, intradermal, intratumoral, intracerebral, intrathecal, and intranasal routes.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, the frequency of administration, the duration of administration monitoring procedures for cell counts, lymphocyte counts, neutrophil counts, and other monitoring information.

Specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material, wherein said pharmaceutical agent comprises a prophylactic or therapeutic agent, and wherein said packaging material includes instruction means which indicate that said agent can be used to prevent, manage, treat, and/or ameliorate one or more symptoms associated with vascular anomalies, or one or more symptoms thereof by administering specific doses and using specific dosing regimens as described herein.

In certain examples, the article of manufacture include labeled antibodies that selectively or specifically bind to embryonic stem cells. As such, the article contains a method to adjust the dosages used in the treatment regimens, and to monitor the efficacy of the regimens.

The present invention provides that the adverse effects that may be reduced or avoided by the methods of the invention are indicated in informational material enclosed in an article of manufacture for use in preventing, treating and/or managing a vascular anomaly. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrohea, myalgia, pain, pruritus, psoriasis, rhinitis, sweating, injection site reaction, and vasodilation.

Further, the information material enclosed in an article of manufacture for use in preventing, treating and/or managing a vascular anomaly can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal oedema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### Kits

The present invention also provides a pharmaceutical pack or kit comprising one or more containers filled with reagents for detecting, monitoring and/or measuring embryonic stem cells associated with vascular anomalies. In one example, the pharmaceutical pack or kit optionally comprises instructions for the use of the reagents provided for detecting and/or measuring embryonic stem cells associated with a vascular anomaly. In another example, the pharmaceutical pack or kit optionally comprises a notice in the form prescribed by a governmental agency regulating the manufacture, use or sale of pharmaceuticals or biological products, which notice reflects approval by the agency of manufacture, for use or sale for human administration.

In an example, the pharmaceutical pack or kit comprises in one or more containers a embryonic stem cell surface marker-binding agent. In certain examples, the agent is an antibody that selectively or specifically binds to an embryonic stem cell surface marker, wherein the embryonic stem cell is associated with a vascular anomaly. The agent may be an antibody (including, e.g., human, humanized, chimeric, monoclonal, polyclonal, Fvs, ScFvs, Fab or F(ab)2 fragments or epitope binding fragments), which cross-reacts with any embryonic stem cell surface marker. In accordance with this example, the pharmaceutical pack or kit comprises one or more antibodies which bind to an embryonic stem cell surface marker, wherein each antibody binds to a different epitope of the embryonic stem cell surface marker and/or binds to the embryonic stem cell surface marker with a different affinity.

For antibody based kits, the kit can comprise, for example: (1) a first antibody (which may or may not be attached to a solid support) which binds to a embryonic stem cell surface marker protein; and, optionally, (2) a second, different antibody which binds to either the embryonic stem cell surface marker protein bound by the first antibody, or the first antibody and is conjugated to a detectable label (e.g., a fluorescent label, radioactive isotope or enzyme). The antibody-based kits may also comprise beads for conducting an immunoprecipitation. Each component of the antibody-based kits is generally in its own suitable container. Thus, these kits generally comprise distinct containers suitable for each antibody. Further, the antibody-based kits may comprise instructions for performing the assay and methods for interpreting and analysing the data resulting from the performance of the assay.

For nucleic acid microarray kits, the kits generally comprise (but are not limited to) probes specific for certain genes attached to a solid support surface. In other examples, the probes are soluble. In one such example, probes can be either oligonucleotides or longer length probes including probes ranging from 150 nucleotides in length to 800 nucleotides in length. The probes may be labeled with a detectable label. The microarray kits may comprise instructions for performing the assay and methods for interpreting and analysing the data resulting from the performance of the assay. The kits may also comprise hybridization reagents and/or reagents necessary for detecting a signal produced when a probe hybridises to a stem cell surface marker nucleic acid sequence. Generally, the materials and reagents for the microarray kits are in one or more containers. Each component of the kit is generally in its own suitable container.

For Quantitative RT-PCR, the kits generally comprise pre-selected primers specific for certain embryonic stem cell surface marker nucleic acid sequences. The Quantitative RT-PCR kits may also comprise enzymes suitable for amplifying nucleic acids (e.g., polymerases such as Taq), and deoxyribonucleotides and buffers needed for the reaction mixture for amplification. The Quantitative RT-PCR kits may also comprise probes specific for the nucleic acid sequences associated with or indicative of a condition. The probes may or may not be labelled with a fluorophore. The probes may or may not be labelled with a quencher molecule. In some examples, the Quantitative RT-PCR kits also comprise components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative RT-PCR kit is generally in its own suitable container. Thus, these kits generally comprise distinct containers suitable for each individual reagent, enzyme, primer and probe. Further, the quantitative RT-PCR kits may comprise instructions for performing the assay and methods for interpreting and analysing the data resulting from the performance of the assay.

A kit can optionally further comprise a predetermined amount of an isolated stem cell surface marker polypeptide or a nucleic acid encoding a stem cell surface marker, e.g., for use as a standard or control. The diagnostic methods of the present invention can assist in conducting or monitoring a clinical study. In accordance with the present invention, suitable test samples, e.g., of serum or tissue, obtained from a subject can be used for diagnosis.

Based on the results obtained by use of the pharmaceutical pack or kit (i.e. whether the stem cell amount has stabilized or decreased), the medical practitioner administering the therapy or regimen may choose to continue the therapy or regimen. Alternatively, based on the result that the stem cell amount has increased, the medical practitioner may choose to continue, alter or halt the therapy or regimen.

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to”.

The invention is further described with reference to the following examples. It will be appreciated that the invention as claimed is not intended to be limited in any way by these examples.

### EXAMPLES

### Example 1: Materials and Methods—Pyogenic Granuloma

### Immunohistochemical Staining

Immunohistochemical (IHC) staining was performed on 4 μm-thick formalin-fixed paraffin-embedded sections of PG samples from 11 patients and proliferating IH samples from 6 patients, in a study approved by the Central Regional Health and Disability Ethics Committee. All IH samples used in this study were confirmed by the expression of GLUT-1 (data not shown).

Antigen retrieval was performed with sodium citrate (Leica, Sydney, Australia), followed by 3,3 diaminobenzidine (DAB) staining with primary antibodies, SOX2, 1:500 (Thermofisher Scientific, CA, USA); phosphorylated STAT3 (pSTAT3), 1:100 (Cell Signalling Technology, MA, USA); OCT4, 1:30 (Cell Marque, CA, USA); NANOG, 1:1000 (Cell Signalling Technology); ERG, 1:200 (Cell Marque); and CD34, ready-to-use (Leica). Primary antibodies were detected using the Bond polymer refine detection kit (Leica), using our previously described protocol.³³

Representative sections of PG were used for immunofluorescent (IF) IHC staining employing the same primary antibodies, but with detection using a combination of Vectafluor Excel anti-rabbit 594 (ready to use; cat# VEDK-1594, Vector Laboratories, CA, USA) and Alexa Fluor anti-mouse 488 (1:500, cat#A21202, Life Technologies, CA, USA) to detect combinations that included pSTAT3, SOX2 and NANOG; and Vectafluor Excel anti-mouse (ready to use, cat# VEDK2488, Vector Laboratories) and Alexa Fluor anti-rabbit 594 (1:500, cat# A21207, Life Technologies) to detect combinations that included OCT4 and CD34.

All antibodies were diluted in Bond primary diluent (Leica), and all DAB and IF IHC staining were performed on the Leica Bond Rx auto-stainer (Leica) and mounted using either Surgipath micromount mounting medium (Leica) or Vectashield hardset mounting medium with DAPI (Vector Laboratories, CA, USA), respectively. Human seminoma tissue sections were used as positive controls.

### NanoString Gene Analysis

Snap-frozen PG samples from the original cohort of 11 patients were used to isolate total RNA for NanoString nCounter™ Gene Expression Assay (NanoString Technologies, Seattle, USA). RNA was extracted from frozen tissues using RNeasy Mini Kit (Qiagen), and quantitated by the NanoDrop 2000 Spectrophotometer (Thermo Scientific), and was subjected to the NanoString nCounter™ gene expression assay performed by New Zealand Genomics Ltd (Dunedin, NZ), according to the manufacturer's protocol. Probes for the genes encoding OCT4 (NM_002701.4), NANOG (NM_024865.2), STAT3 (NM_139276.2) and the housekeeping gene GUSB (NM_00181.3) were designed and synthesized by NanoString Technologies. Raw data were analyzed by nSolver™ software (NanoString Technologies) using standard settings and were normalized against the housekeeping gene.

### In-Situ Hybridization

Representative 4 μm-thick formalin-fixed paraffin-embedded PG sections (n=3) from the original cohort of 6 patients were used for mRNA in-situ hybridization (ISH) staining, on the Leica Bond Rx autostainer and detected using the ViewRNA red stain kit (Affymetrix, CA, USA) as previously described.³³ The probes used for OCT4, NANOG, SOX2 and STAT3 were obtained from Affymetrix. Human seminoma tissue sections were used as positive controls.

### Image Analysis

All IF IHC images were captured using the Olympus FV1200 confocal microscope (Tokyo, Japan) and all DAB IHC images were captured using the Olympus BX53 bright field microscope fitted with an Olympus DP21 digital camera (Tokyo, Japan).

### Example 2: Results—Pyogenic Granuloma

### Immunohistochemical Staining

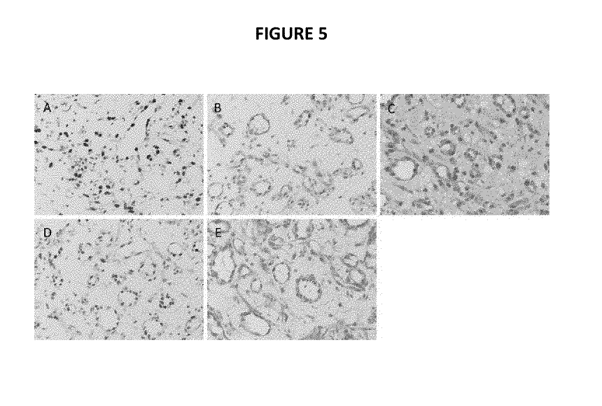

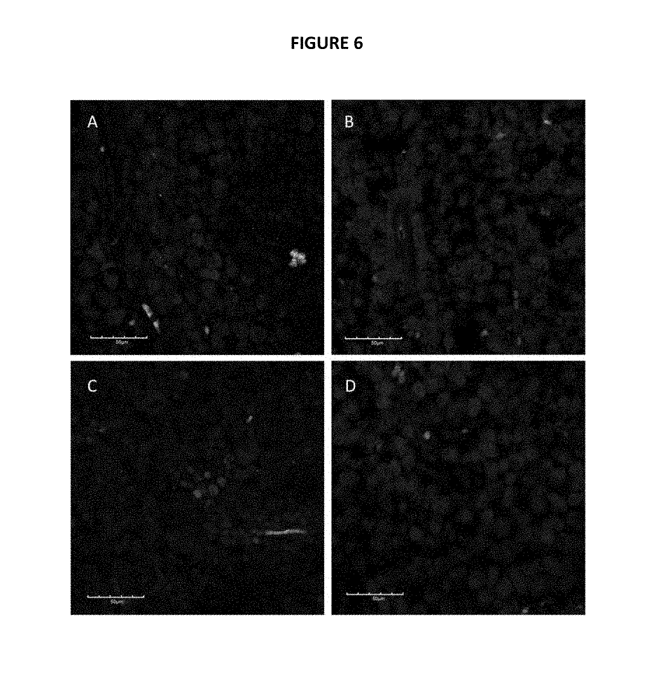

DAB IHC staining confirmed the expression of the endothelial marker ERG⁸² of the microvessels of PG (FIG. 5A, brown), as well as the embryonic stem cell markers OCT4 (FIG. 5B, brown), SOX2 (FIG. 5C, brown), pSTAT3 (Suppl FIG. 1D, brown) and NANOG (FIG. 5E, brown). The expression of the corresponding markers was similarly seen in the microvessels of proliferating IH lesions (FIG. 6 A-E, brown).

IF IHC staining showed the microvessels of PG were composed of an endothelial layer that expressed both ERG (FIG. 1A, red) and CD34 (FIG. 1A, green). Human seminoma tissue was used as a positive control for IF IHC staining for OCT4 (FIG. 6A, green), SOX2 (FIG. 6B, red), pSTAT3 (FIG. 6C, red), and NANOG (FIG. 6D, red).

OCT4 (FIG. 1B, green) was co-expressed on the endothelium of PG expressing ERG (FIG. 1B, red). SOX2 (FIG. 1C, red), a key binding partner of OCT4,⁸³ was also demonstrated on the endothelium expressing CD34 (FIG. 1C, green, arrowheads) as well as cells in the interstitium (FIG. 1C, arrows). The expression of pSTAT3 (FIG. 1D, red), the activator of genes essential for ESC⁸⁴, was localized to the endothelium (FIG. 1D, red, arrowheads) and cells in the interstitium (FIG. 1D, red, arrows) of PG, identified by CD34 staining (FIG. 1D, green). NANOG (FIG. 1E, red, arrows), another transcription factor required to maintain pluripotency of ESC,⁷² was expressed by the endothelial cells identified by CD34 (FIG. 1E, green), as well as some cells within the interstitium (FIG. 1E, red, arrows).

### NanoString Gene Analysis

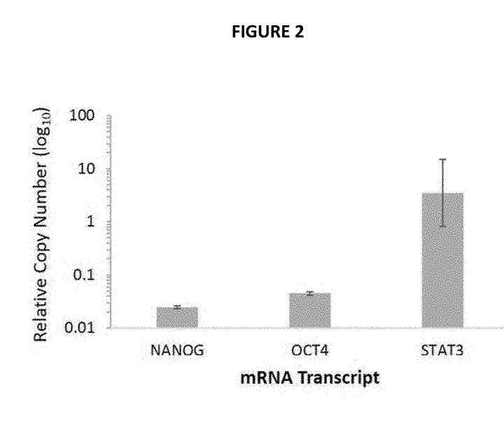

To confirm the translational abundance of the ESC markers identified by IHC staining we used transcriptional profiling of PG samples from the same cohort of six patients used for investigating levels of mRNA. Nano-String analysis (FIG. 2) revealed transcriptional levels of mRNA corresponding to OCT4, NANOG and STAT3 proteins in PG, confirming the IHC findings.

### In-Situ Hybridization

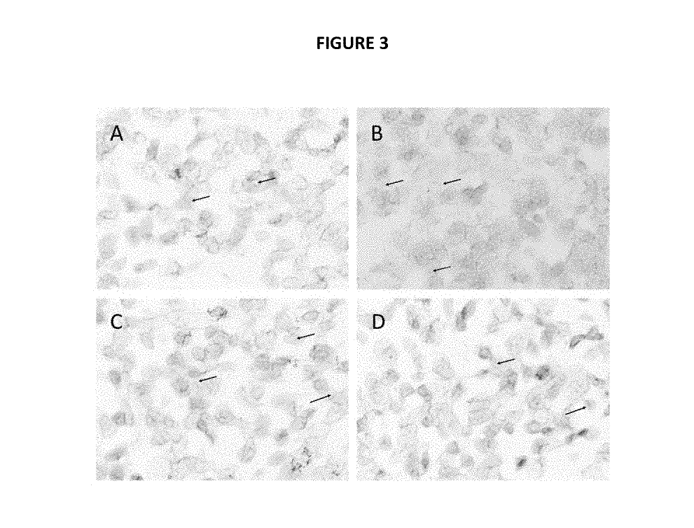

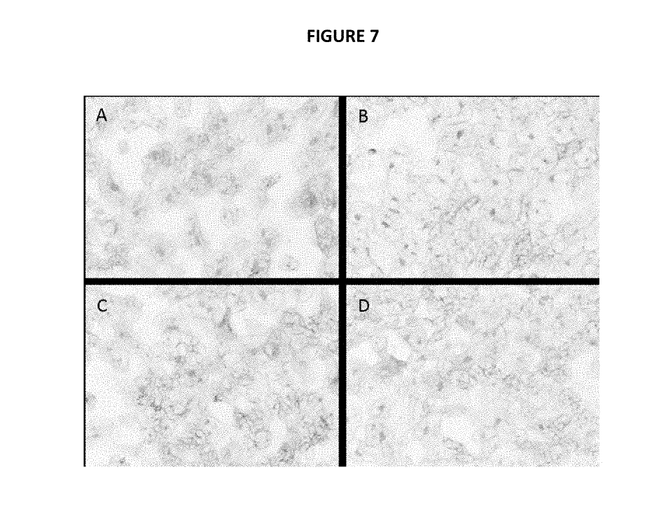

ISH staining confirmed the expression of the mRNA for OCT4 (FIG. 3A, pink, arrows), SOX2 (FIG. 3B, pink, arrows), STAT3 (FIG. 3C, pink, arrows) and NANOG (FIG. 3D, pink, arrows) in the cells on the inner endothelial layer of the microvessels in PG. Human seminoma tissue was used as a positive control for ISH (FIG. 7) for all the ESC markers investigated.

DAB staining of the endothelium of (FIG. 5A, brown) demonstrated that this endothelium also expressed the embryonic stem cell markers OCT4 (FIG. 4, OCT4 & FIG. 5B, brown), SOX2 (FIG. 4, SOX2 & FIG. 5C, brown), pSTAT3 (FIG. 5D, brown) and NANOG (FIG. 5E, brown). The same endothelium also expressed the renin receptor (FIG. 4. RR, brown) and angiotensin converting enzyme (FIG. 4. ACE, brown).

### Example 3: Discussion—Pyogenic Granuloma

Embryonic stem cells are totipotent stem cells that arise from the blastocyst of the early mammalian embryo with the potential for unrestricted proliferation and differentiation. The hallmark of ESC is their ability to form all three germ layers in vitro and teratoma in vivo.⁸⁴

First isolated in 1998,⁶³ there are now over 300 lines of human ESC currently in use.⁶⁵ These cell lines have enabled the discovery of proteins and transcription factors necessary for the maintenance of pluripotency which can be used to identify an ESC phenotype.

Applicants' demonstration of the expression of the ESC markers, NANOG, SOX2 and pSTAT3, on the endothelium of PG suggests that these endothelial cells display a primitive phenotype. STAT3, NANOG and SOX2 interact with OCT4 by a complex network to maintain the pluripotency of ESC. STAT3, a signal transducer and a member of the activator transcription factor family, directly regulates the actions of OCT4, to block the differentiation of cells into more mature lineages in knock-out mice.⁷⁵ Although STAT3⁻/STAT3⁻ mice still form early blastocysts with normal morphology, their embryos fail to produce OCT4⁺ outgrowths and are unable to produce ESCs.⁷⁵

The downstream OCT4 and SOX2 work synergistically to activate genes, including NANOG, to maintain pluripotency.^{71,86} NANOG, a homeobox containing transcription factor, is regulated by the OCT4/SOX2 complex and has been implicated in the self-renewal of undifferentiated ESCs through the activation of the Rex1 promoter region.⁸² In 2007, OCT4, SOX2, NANOG and LIN were used to generate iPSCs from human fibroblasts, underscoring their importance in the production of an ESC-like phenotype.⁷⁶

The expression of these ESC markers on the microvessels of PG infers a primitive endothelial phenotype. As all of these transcription factors act on the nucleus of the cells, nuclear expression of these markers suggests that they are both present and activated within the endothelial cells. Transcriptional activation of the genes for these proteins is supported by the presence of mRNA for OCT4, STAT3 and NANOG as detected by NanoString analysis and supported by the ISH data localizing the mRNA for all four of these ESC markers to the putative primitive endothelium. The hallmark of ESC is the ability to form all three germ layers in vitro and teratoma in vivo, which is not observed in PG. Applicants infer that these primitive cells are downstream of ESC, similar to those identified within IH.⁸⁰ Interestingly OCT4 was the only ESC transcription factor that was predominantly expressed in the cytoplasm of the endothelial cells, as was also observed in proliferating IH⁸⁰ and in bladder tumors.⁸⁷

The expression of NANOG in both the endothelium of the microvessels and some cells within the interstitium, is intriguing. This indicates the presence of two separate populations of primitive cells within PG, as observed in proliferating IH.⁸⁰ These findings suggest either the NANOG cells within the interstitium of PG are downstream derivatives of the more primitive endothelium or that there are two independent primitive populations. The absence of the expression of OCT4 by the interstitial cells, however, supports the notion that these are more downstream cells, potentially arising from the primitive endothelium.⁸⁰ It is tempting to speculate that the primitive endothelium could be the origin of PG, as an aberrant proliferation of microvessels by de novo vasculogenesis originating from these primitive embryonic-like stem cells. This novel finding offers potential insights into the pathogenesis of PG, as a developmental anomaly arising from the primitive mesoderm.⁸⁹ It is exciting to postulate that the non-uniform expression of these ESC markers in the endothelium of the microvessels may indicate more mature cells budding off the putative primitive endothelium, potentially resulting in a hierarchical expression pattern of ESC markers observed in PG.

### Example 4: Methods and Materials—Venous Malformation

### Tissue Collection

SC VM tissues from 7 patients aged 9-30 (mean, 22.2) years and IM VM from 7 patients aged 16 months to 54 years (mean, 21.3 years) were sourced from the Gillies Mclndoe Research Institute Tissue Bank, in accordance with a protocol approved by the Central Health & Disability Ethics Committee (ref. no. 13/CEN/130). The tissues used in this study were confirmed to be SC or IM VM by a consultant anatomical pathologist using H&E staining and negative D2-40 staining to rule out lymphatic malformation (see below).

### Immunohistochemical Staining

Immunohistochemical (IHC) 3,3-diaminobenzidine (DAB) staining was performed on 4 μm-thick formalin-fixed paraffin-embedded sections of VM samples as reported previously.³³ In brief IHC staining was performed using the Leica Bond Rx auto-stainer (Leica, Australia) with antibodies against CD34 (ready to use; cat# PA0212, Leica), D2-40 (1:100; cat# M3619, Dako), PRR (1:100, cat# HPA003156, Sigma), ACE (1:40; cat#3C5, Serotec), ATIIR1 (1:25; cat# ab9391, Abcam), ATIIR2 (1:2000; cat# NBPI-77368, Novus), OCT4 (1:200; cat# NBPI-47923, Novus Biologicus, CO, USA), NANOG (1:1000; cat# NBPI-04320, Novus Biologicus), SOX2 (1:500, cat# PA1-094, Thermo Fisher Scientific, CA, USA) and pSTAT3 (1:100; cat# D3A7, Cell Signalling, MA, USA). All antibodies were diluted in Bond primary diluent (Leica).

Positive control samples were selected based on previously reported expression of the relevant protein: placenta for PRR³⁴, kidney for ACE³⁵ and ATIIR2³⁶, and liver for ATIIR1³⁷. To determine the specificity of the primary antibodies, we performed staining on a VM tissues by omitting the primary antibodies.

### Image Analysis

All DAB IHC stained slides were viewed and the images were captured using the Olympus BX53 microscope fitted with an Olympus DP21 digital camera (Olympus, Tokyo, Japan).

### NanoString Gene Expression Analysis

Total RNA was extracted from 10 mg of snap-frozen IM (n=3) and SC (n=3) VM tissues from the same cohort of patients included in IHC staining, using RNeasy Mini Kit (Qiagen), and quantitated with the NanoDrop 2000 Spectrophotometer (Thermo Scientific). The samples with A260/A230≧8 and A260/A280≧9 were used for further analysis. The integrity of the RNA was assessed by Agilent 2100 BioAnalyser (Agilent Technologies). The isolated RNA was then subjected to NanoString nCounter™ Gene Expression Assay (NanoString Technologies, Seattle, USA) as completed by New Zealand Genomics Ltd (Dunedin, NZ), according to the manufacturer's protocol. Probes for the genes encoding ATP6AP2 (NM_005765.2), ACE (NM_000789.2), ATIIR1 (NM_000686.3), ATIIR2 (NM_000685.3), and the housekeeping gene, GAPDH (NM_002046.3) were designed and synthesized by NanoString Technologies.

### Statistical Analyses

Raw NanoString data was analyzed using SPSS (v22, IBM), validated with nSolver™ software (NanoString Technologies) using standard settings, and normalized against the housekeeping gene.

To determine statistical significance between the IM and SC samples, two-tailed student's t-test was performed. Charts were made with Excel.

### Example 5: Results—Venous Malformation

### Immunohistochemical Staining

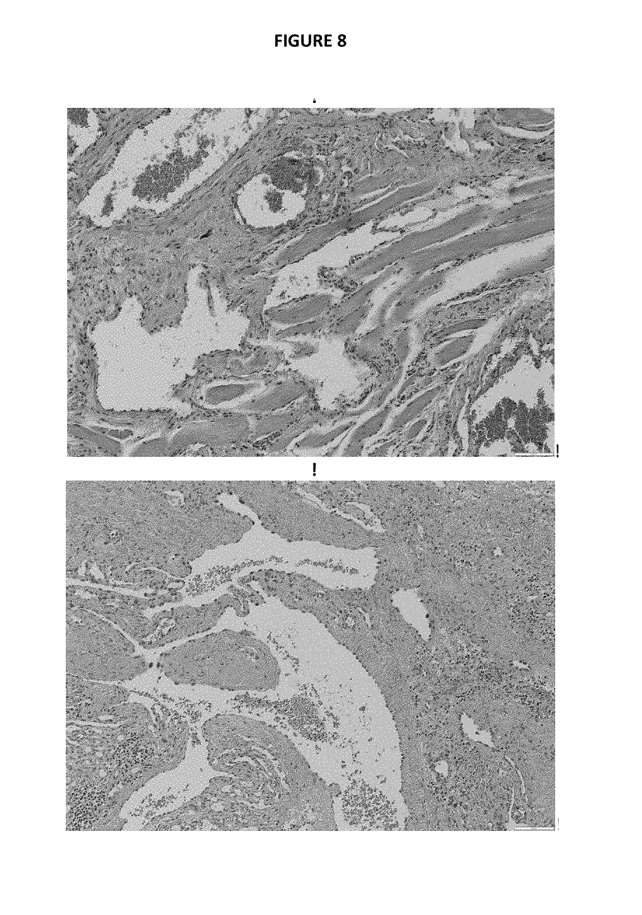

Identification of the vasculature within the VM lesions was achieved with H&E staining, characterised by thin-walled ectatic venous channels with deficient or absent SMC in both IM (FIG. 8 top panel) and SC (FIG. 8 bottom panel)

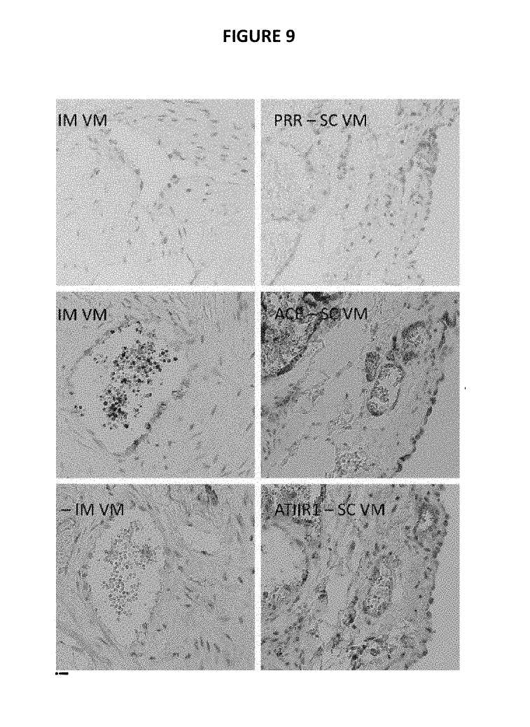

PRR was expressed on the endothelium of both IM (FIG. 9 top left panel, brown) and SC (FIG. 9 top right panel, brown) VM. The expression of ACE, which converts ATI to ATII, was demonstrated on the endothelium of IM (FIG. 9 middle left panel, brown) and SC (FIG. 9 middle right panel, brown) VM. To demonstrate a potential receptor available for downstream signaling of ATII, ATIIR1 and ATIIR2 were stained for in our cohort. This demonstrated the expression of ATIIR1 on the endothelium of VM vessels in IM (FIG. 9 bottom left panel, brown) and SC (FIG. 9 bottom right panel, brown) VM. ATIIR2 was not detected in any of the IM or SC VM lesions studied.

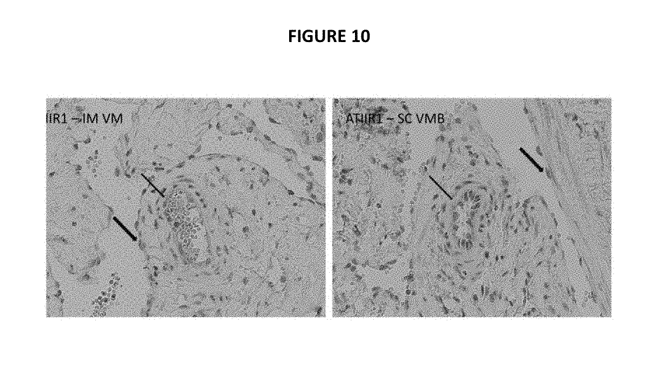

There was more intense staining for ATIIR1, in both IM and SC VM, in the smaller lesional vessels (FIG. 10, brown, thin arrows) compared to the larger vessels (FIG. 10, brown, thick arrows).

Positive controls for PRR (FIG. 14A, brown), ACE (FIG. 14B, brown), ATIIR1 (FIG. 14C, brown), ATIIR2 (FIG. 14D, brown) and the stain with omission of the primary antibody (FIG. 14E, brown) demonstrated appropriate specificity for the antibodies used.

### NanoString Gene Analysis

NanoString gene analysis was used to evaluate and quantify the presence of the components of the RAS within VM at the transcription level. mRNA corresponding to PRR, ACE and ATIIR2, but not ATIIR1 was detected in VM tissue samples. Expression of ATP6AP2 was higher in SC samples than in IM, this difference was shown to be statistically significant (p≦0.05).

### Example 6: Discussion—Venous Malformation

The results presented in Example 5 demonstrate the presence of PRR, ACE, and ATIIR1, but not ATIIR2 on the endothelium of the lesional vessels in SC and IM VM at both the mRNA and protein levels. IHC analysis of the SC IM samples revealed relatively increased staining patterns for ATIIR1 on the endothelium of smaller lesional vessels compared to larger lesional vessels, although there was no difference at the transcriptional activation level.

The presence of PRR, ACE, and ATIIR1 on the endothelium of VM indicates a putative role for the RAS. Nguyen Dinh Cat et al.²⁷ have demonstrated the components of the RAS including PRR, ACE ATIIR1 and ATIIR2 in normal vascular tissues.

ATIIR1 and ATIIR2 have been demonstrated to serve distinct functions, but share ATII as a common ligand.³⁸ This would therefore suggest that the absence of ATIIR2 may lead to preferential signalling down the ATIIR1 pathway. As ATIIR1 is responsible for the pro-angiogenic effects of ATII,³⁹ this may, in part, explain the increased density of abnormal venous channels in these lesions. ACE is expressed on the endothelium of the SC and IM VM samples examined in this study. ACE has been shown to be a marker for identifying primitive hemangioblasts derived from human pluripotent stem cells, as well as contributing to their regulation.⁴⁰ Zambidis et al.⁴⁰ demonstrate preferential differentiation down an endothelial lineage via ATIIR1 signalling. In the presence of ATIIR1 and the absence of ATIIR2 differentiation is likely to be directed preferentially down the endothelium rather than pericyte lineage.

PRR possesses a variety of actions, including binding pro-renin and renin, and downstream signalling through MAP kinases ERK1 and ERK2.³⁴ Pro-renin bound to PRR undergoes a conformational change, exposing the active sites and rendering it enzymatically active.⁴¹ Renin bound to PRR has a 4-fold increase in catalytic activity.³⁴ The presence of PRR in the endothelium of VM indicates a potential role for facilitating local conversion of angiotensinogen to ATI.

Also in support of the involvement of stem cells in VM is an article by Mogler et al.⁴⁵ reporting positive cytoplasmic staining for the stem cell growth factor receptor (c-kit) in the small lesional vessels, but not in larger vessels, of VM. We find a similar pattern of stronger staining for ATIIR1 in the endothelium of smaller lesional vessels. It is exciting to speculate that the absence of c-kit in larger lesional vessels may indicate that these dilated vessels can no longer maintain a putative stem cell population. However, this is a topic of our ongoing research.

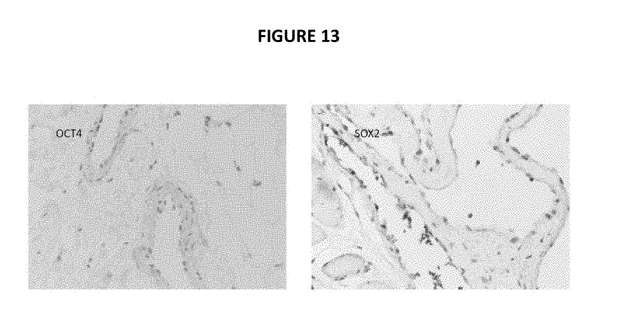

A previous study has shown that ATII, acting through ATIIR1, stimulates the production of angiopoietin-2 (Ang-2), a well-known ligand for TIE2.⁴⁶ Research into the effects of Ang-2 on TIE2 has revealed that Ang-2 acts to modulate the effect of angiopoietin-1 (Ang-1) on TIE2,⁴⁷ as well as increasing the effects of VEGF.⁴⁸ It is therefore possible that Applicants findings suggesting increased ATIIR2 signalling can lead to increased production of Ang-2, and cause the development of VM through TIE2, a mutation implicated in the pathogenesis of VM.⁸ FIG. 13 shows a schematic representation of Applicants proposed model.

Current treatments of VM aim to relief symptoms caused by VM, rather than halting and reversing the process leading to development and expansion of these lesions. These treatments have limited efficacy,¹⁴ and depending on the location of the lesion, may not be feasible.

This is the first report to demonstrate the presence of PRR, ACE and ATIIR1, the components of RAS, on the endothelium of SC and IM VM at both the transcriptional and translational levels. Applicants infer a role of the ATII ligand in biology signalling of VM and highlights a potential role of the inhibitors of PRR, ACE and ATIIR1 in the treatment of VM.

### Example 7: Methods & Materials #2—Venous Malformation

### Tissue Samples

Previously untreated, SCVM tissue samples from seven patients and IMVM samples from seven patients with a mean age of 22.9 (range, 1.2-54) and 21.1 (range, 8-30) years, respectively, were sourced from the Gillies Mclndoe Research Institute Tissue Bank, and used in a study approved by the Central Health & Disability Ethics Committee (ref. no. 13/CEN/130). Written informed consent was obtained from the participants.

### Histology and Immunohistochemical Staining

Hematoxylin and eosin (H&E) staining was performed on 4 μm-thick formalin-fixed paraffin-embedded sections of SCVM (n=7) and IMVM (n=7) samples to confirm the presence of VM tissues on the slides by an anatomical pathologist (HDB). Negative staining for D2-40 (1:100; cat# M3619, Dako, Glostrup, Denmark) was used to exclude lymphatic malformation.

3,3-Diaminobenzidine (DAB) immunohistochemical (IHC) staining for primary antibodies Nanog (1:100: cat# ab80892, Abcam, Cambridge, UK), pSTAT3 (1:100; cat#9145, Cell Signaling Technology, Danvers, Mass., USA), OCT4 (1:30; cat# MRQ-10, Cell Marque, Santa Cruz, Calif., USA), SOX2 (1:200, cat# PA1-094, Thermo Fisher Scientific, Waltham, Mass., USA), SALL4 (1:30; cat# CM385M-16, Cell Marque, Rocklin, Calif., USA) and CD44 (1:1500; cat# MRQ-13, Cell Marque), was performed on the SCVM and IMVM tissue sections using the Leica Bond Rx auto-stainer (Leica), as previously described.⁹⁰ Nanog was stained using the ImmPACT NovaRED Peroxidase Substrate Kit (cat# SK-4805, Vector Laboratories, Burlingame, Calif., USA) and the ImmPRESS Excel Amplified HRP Polymer Staining Kit (cat# MP-7601, Vector Laboratories). To confirm co-expression of two proteins, immunofluorescent (IF) IHC staining with the same primary antibodies at the same concentrations was performed with CD34 (ready-to-use; cat# PA0212, Leica, Newcastle-Upon-Tyne, UK) and ERG (1:200; cat# EP111, Cell Marque), as appropriate endothelial markers. Appropriate secondary antibodies, Vectafluor Excel anti-rabbit 594 (ready-to-use; cat# VEDK-1594, Vector Laboratories, Burlingame, Calif., USA) and Vectafluor Excel anti-mouse (ready-to-use; cat# VEDK2488, Vector Laboratories) combinations were used for IF IHC detection. All antibodies were diluted with Bond TM primary antibody diluent (cat# AR9352, Leica).

Positive human controls tissues used were seminoma for Nanog, SALL4 and OCT4; skin for SOX2; and tonsil for pSTAT3 and CD44.^{91,92} To determine the specificity of the primary antibodies, appropriate negative controls consisting of combined Flex Negative Control Mouse (ready-to-use; cat# IR750, Dako, Carpinteria, Calif., USA) and Flex Negative Control Rabbit (ready-to-use; cat# IR600, Dako) staining was performed on VM tissues.

### Microscopy

All DAB IHC-stained slides were viewed and the images were captured using an Olympus BX53 light microscope fitted with an Olympus DP21 digital camera (Tokyo, Japan). IF IHC-stained slides were viewed and the images were captured using an Olympus FV1200 biological confocal laser-scanning microscope (Tokyo, Japan).

### NanoString Gene Analysis

NanoString gene analysis was performed on snap-frozen samples of SCVM (n=3) and IMVM (n=3) from the respective original cohort of patients included in IHC staining, as previously described.⁹³ Probes for the genes encoding STAT3 (NM_139276.2), OCT4 (NM_002701.4), CD44 (NM_001001392.1) and the housekeeping gene GAPDH (NM_002046.3), were designed and synthesized by NanoString Technologies (Nanostring Technologies, Seattle, Wash., USA)

NanoString data was analyzed using SPSS (v22, IBM), and validated with nSolver™ software (NanoString Technologies) using standard settings, normalized against the housekeeping gene. Charts were made with Excel.

### RT-qPCR

Total RNA was isolated from formalin-fixed paraffin-embedded samples of SCVM (n=3) and IMVM (n=3) from the original cohorts of patients included for DAB IHC staining, using the RNeasy FFPE Kit (cat#73504, Qiagen, Hilden, Germany) with DNase digest and the QIAcube system (Qiagen). Total RNA quantity and quality were determined using NanoDrop 2000 (Thermo Fisher Scientific, Waltham, Mass., USA). Reverse transcription reactions were performed using the iScript Reverse Transcription Supermix (Bio-Rad, Hercules, Calif., USA). The expression of stem cell markers was detected using gene-specific TaqMan (Thermo Fisher) primers probes (SOX2: Hs01053049_s1; SALL4: Hs00360675_m1; Nanog: Hs04399610_g1; GAPDH: 4333764T) with the Rotor-Gene Multiplex RT-PCR Kit (cat#204974, Qiagen). All measurements were performed in duplicate. Gene expression was determined by the Relative Standard Curve Method, using GAPDH as an endogenous control. Graphs were generated with Microsoft Excel and results are shown as relative expression.

### Statistical Analysis

The mean levels of mRNA expression for each gene investigated in SCVM vs IMVM were subjected to t-test for quality of means using SPSS v 22, to determine and significant differences.

### Example 8: Results #2—Venous Malformation

Histology and 3,3-diaminobenzidine Immunohistochemical Staining

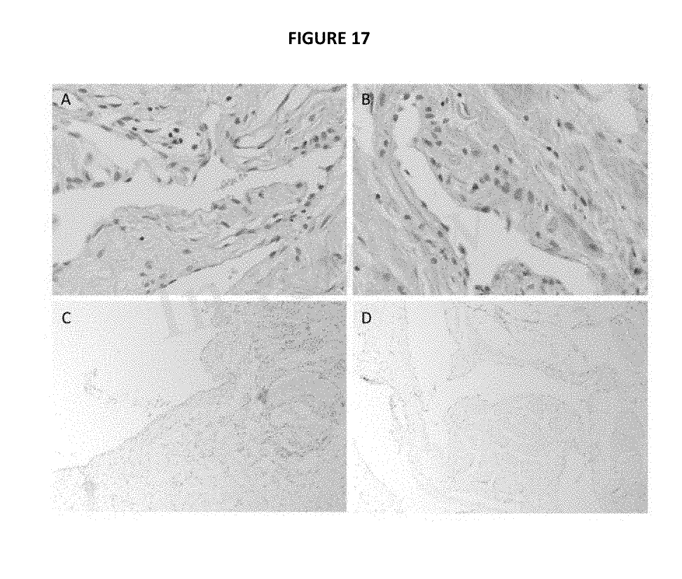

VM tissues, characterized by ectatic venous channels in both SCVM (FIG. 17A) and IMVM (FIG. 17B), were identified by H&E staining. None of the SCVM (FIG. 17C, brown) and IMVM (FIG. 17D, brown) lesions used in this study expressed D2-40, a lymphatic marker.

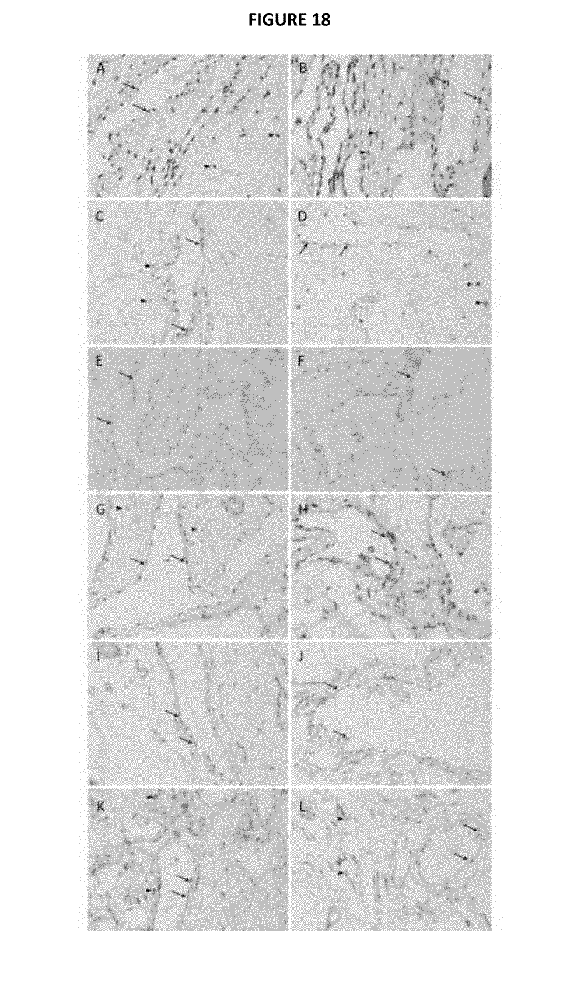

Nanog (FIGS. 18 A&B, red), pSTAT3 (FIGS. 18 C&D, brown), OCT4 (FIGS. 18 E&F, brown), SOX2 (FIGS. 18 G&H, brown), SALL4 (FIGS. 18 I&J, brown) and CD44 (FIGS. 18 K&L, brown) were expressed on the endothelium of all seven samples of SCVM (FIGS. 18 A, C, E, G, I & K) and seven samples of IMVM (FIGS. 18 B, D, F, H, J & L). Interestingly, cells away from the endothelium also expressed Nanog (FIGS. 18 A&B, red, arrowheads), pSTAT3 (FIGS. 18 C&D, brown, arrowheads), SOX2 (FIGS. 18 G&H, brown, arrowheads) and CD44 (FIGS. 18 K&L, brown, arrowheads) in all samples of SCVM (FIGS. 18 A, C, G & K) and IMVM (FIGS. 18 B, D, H & L).

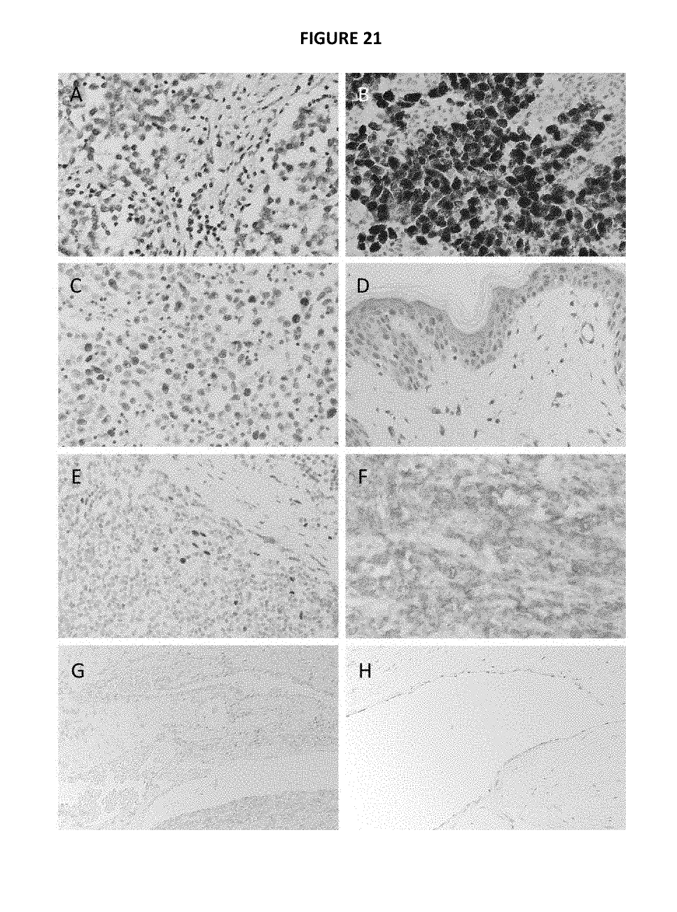

Positive staining was demonstrated in seminoma for Nanog (FIG. 21A, red), OCT4 (FIG. 21B, brown) and SALL4 (FIG. 21C, brown); skin for SOX2 (FIG. 21D, brown); and tonsil for pSTAT3 (FIG. 21E, brown) and CD44 (FIG. 21F, brown). Negative control SCVM (FIG. 21G, brown) and IMVM (FIG. 21H, brown) tissue samples demonstrated minimal staining.

### Immunofluorescent Immunohistochemical Staining

IF IHC staining with CD34 (FIGS. 19 A&B, green) and ERG (FIGS. 19 A&B, red) demonstrated CD34⁺/ERG⁻ (long arrows), CD34⁺/ERG⁺ (short arrows), and CD34⁻/ERG⁺ (arrowheads) endothelium in SCVM (FIG. 19A) and IMVM (FIG. 19B) lesions. The CD34⁺ (FIGS. 19 C&D, green) endothelium expressed Nanog in both SCVM (FIG. 19C, red, arrows) and IMVM (FIG. 19D, red, arrows) lesions with cells away from endothelium also expressing Nanog (FIGS. 19 C&D, red, arrowheads) within SCVM (FIG. 19C) and IMVM (FIG. 19D) lesions. The CD34⁺ (FIGS. 19 E&F, green) endothelium expressed pSTAT3 in both SCVM (FIG. 19E, red) and IMVM (FIG. 19F, red, arrows) lesions with cells away from the endothelium also expressing pSTAT3 (FIGS. 19 E&F, red, arrowheads), within SCVM (FIG. 19E) and IMVM (FIG. 19F) lesions. The ERG⁺ (FIGS. 19 G&H, red) endothelium expressed OCT4 in SCVM (FIG. 19G, green, arrows) and IMVM (FIG. 19H, green, arrows) lesions with cells outside of the endothelium stained negatively for OCT4. The CD34⁺ (FIGS. 19 I&J, green) endothelium expressed SOX2 in SCVM (FIG. 19I, red, arrows) and IMVM (FIG. 19J, red, arrows) lesions with cells away from the endothelium also expressing SOX2 (FIGS. 19 I&J, red, arrowheads) within SCVM (FIG. 19I) and IMVM (FIG. 19J) lesions. The ERG⁺ (FIGS. 19K&L, red) endothelium expressed SALL4 in SCVM (FIG. 19K, green, arrows) and IMVM (FIG. 19L, green, arrows) lesions with no expression of SALL4 on the cells outside of the endothelium. The ERG⁺ (FIGS. 19 M&N, red) endothelium expressed CD44 in SCVM (FIG. 19M, green, arrows) and IMVM (FIG. 19N, green, arrowheads) lesions with cells away from the endothelium also expressing CD44 (FIGS. 19 M&N, green, arrowheads) in SCVM (FIG. 19M) and IMVM (FIG. 19N) lesions. Dual IF IHC staining showed co-expression of Nanog (FIGS. 19 O&P, red, arrows) and CD44 (FIGS. 19 O&P, green, arrows) in cells outside of the endothelium within SCVM (FIG. 19O) and IMVM (FIG. 19P) lesions, inferring the non-endothelial Nanog⁺ cells and the non-endothelial CD44⁺ cells are a single population.

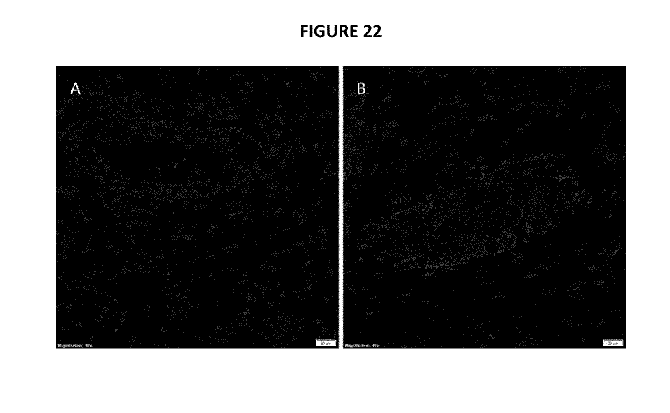

Individual IF IHC staining for each of the aforementioned proteins are presented in Supplemental FIGS. 18 & 19 for SCVM and IMVM, respectively. Negative controls for IF IHC staining for both SCVM and IMVM tissue samples demonstrated minimal staining (FIG. 22).

### NanoString Gene Analysis

NanoString transcriptional profiling of three SCVM and three IMVM samples were normalized against the housekeeping gene GAPDH and averaged confirming the relative abundance of mRNA transcripts for STAT3 and CD44 in all SCVM and IMVM (FIG. 20A). Statistical analysis of the mean values revealed no significant differences between the expression of STAT3 and CD44 between the SCVM and IMVM samples.

### RT-qPCR

Average expression levels of SOX2, SALL4 and Nanog genes, relative to the housekeeping gene GAPDH, are shown in FIG. 20B. There were no significant differences between the mean expression levels of SOX2, SALL4 and Nanog between SCVM and IMVM samples.

### Example 9: Discussion #2—Venous Malformation

The pathogenesis of VMs is yet to be elucidated. 98.8% of VMs arise sporadically. Familial VM that is typically multifocal is inherited in an autosomal dominant manner.⁹⁴⁻⁹⁸ The activating mutations of the tyrosine kinase receptor, TIE2, in the ECs, accounts for the familial forms and 50% of sporadic^{94,95,99,97} VMs. The most common mutation in familial VM is R849W that involves an arginine-to-tryptophan substitution at position 849 in the kinase domain of TIE2.^{94,96,97,98} The most common somatic mutation is L914F, which accounts for 77% of patients with mutation-positive VM.⁹⁷

The exact mechanism by which mutant TIE2 leads to VMs is unknown.⁹⁴ The mutations which lead to VM are located in the tyrosine kinase domain, kinase-insert domain, and carboxy terminal tail domains, and cause ligand-independent receptor hyperphosphorylation in vitro and increased TIE2 activity.^{94,96,97,98} The lack of correlation between phosphorylation and strength and severity of patient phenotype suggests a role in qualitative and not just quantitative anomalies in TIE2 signaling.⁹⁴ The activating TIE2 mutation in ECs may reduce SMC ligand expression causing a local uncoupling between the normal recruitment of SMCs and the proliferation of ECs, resulting in affected vessels containing a disproportionately large number of ECs compared to SMCs.⁹⁸

Studies on mutant TIE2 have shown that expression of TIE2-L914F or TIE2-R849W in HUVECs increased activation of AKT and of STAT-1, an inflammatory mediator.^{94,97} Elevated AKT signaling has an anti-apoptotic effect on ECs leading to increased survival, as well as reducing the production of PDGF-B, which plays a major role in recruitment of mural cell.^{94,97} Increased activity of this receptor tyrosine kinase that leading to abnormal sprouting and branching, which results in VMs, has been proposed.⁹⁶ Vascular endothelial protein, tyrosine phosphatase, which is more strongly expressed in vessels invested with SMC than in capillaries and small veins, has been suggested to protect arteries and large veins from increased TIE2 activity, resulting in malformed venules/veins.⁹⁶

TIE2 is expressed on ECs, hematopoietic stem cells and proangiogenic monocytes.⁹⁷ Further research has identified ligands for the TIE2 receptor, Ang-1 and angiopoietin-2 (Ang-2).^{94,97} Ang-1 and Ang-2 bind to TIE2 and mediate, respectively, vascular maturation and angiogenesis.⁹⁷ The TIE2 signaling pathway, through these proteins, is critical for EC-SMC communication in venous morphogenesis and believed to play a role in regulating the assembly of non-endothelial components of the vessel including SMCs.^{95,96} Knockout of TIE2 or Ang-1 in mice results in impaired blood vessel branching and deficient perivascular coverage.⁹⁷ Deletion of Ang-1 in the developing embryo produces a disorganized vascular network with an increased number of ectatic vessels.⁹⁷

Involvement of activation of Tie2 receptor in the development of dilated luminal vessels derived from ESC.¹⁰⁰ This led us to infer the putative presence of a primitive population within VM in the development of these ectatic vessels.

In this study, we have demonstrated expression of Nanog, pSTAT3, OCT4, SOX2, SALL4, and CD44 in the endothelium of both SCVM and IMVM. We have also demonstrated that Nanog, pSTAT3, SOX2 and CD44 are also expressed by cells outside of the endothelium, potentially by the same cells. These findings suggest the presence of at least two ESC-like subpopulations, one within and one outside of the endothelium of both SCVM and IMVM. Given that the endothelial ESC-like subpopulation expresses the more primitive marker, OCT4¹⁰¹, it is exciting to speculate that they give rise to the non-endothelial population. However, equally it is possible that there may be two distinct ESC-like subpopulations. Further work is needed to determine the relationship between these two ESC-like subpopulations.

This study describes an intriguing combined expression of ESC markers by the endothelium of both SCVM and IM VM lesions. Although some of these transcription factors, such as pSTAT3 may be associated with the normal hematopoiesis¹⁰² we infer that its expression maybe more related to its role in stem cell signaling, we infer that its expression maybe more related to its role in stem cell signaling.¹⁰³ Furthermore the expression of SOX2 and SALL4 are seen in both the cytoplasm and nucleus, which is supported by similar studies^{104,105}, although the reasons for which are beyond the scope of this study. The core nuclear transcription factors Nanog, pSTAT3 and OCT4 have been used to identify and characterize the ESC population.¹⁰⁶ OCT4 works synergistically with SOX2 and Nanog, to regulate various genes required for self-renewal and pluripotency.^{107,108} The presence of leukemia inhibitory factor in pSTAT3 leads to its interaction with brachyury to form a loop stimulating the expression of Nanog.¹⁰⁸

Mogler et al.¹⁰⁹ show the presence of c-kit, a stem cell growth factor receptor, in the smaller, but not larger vessels of VM lesions. A potential explanation could be that the larger ectatic vessels are more mature and can no longer maintain a stem cell population, and the smaller vessels act as potential precursors.

We have recently demonstrated the expression of PRR, a component of the RAS, on the endothelium of both SCVM and IMVM.⁹³ PRR is known to signal through the Wnt/β-catenin pathway¹¹⁰ and subsequently maintain pluripotency in ESCs.¹¹¹

The finding of the two ESC-like subpopulations within SCVM and IMVM is novel and suggests that these primitive subpopulations may be a therapeutic target. Work is underway to investigate if these primitive subpopulations expressing the RAS which can be manipulated by existing medications.

Taken together the novel findings in this report suggest a role of the ESC expression pattern on the TIE2 activating mutation endothelium may possibly predispose to the formation VM phenotype.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Mulliken J B, Glowacki J. Hemangiomas and vascular malformations in infants and children: a classification based on endothelial characteristics. Plast Reconstr Surg [Internet]. 1982 [cited 2014 Nov. 7]; 69(3):412-22. Available from: http://journals.lww.com/plasreconsurg/Abstract/1982/03000/Hemangiomas_and_Vascular_Malformations_in_Infants.2.aspx
2. Eifert S, Villavicencio J L, Kao T-C, Taute B M, Rich N M. Prevalence of deep venous anomalies in congenital vascular malformations of venous predominance. J Vasc Surg [Internet]. 2000 March [cited 2014 Nov. 12]; 31(3):462-71. Available from: htt://linkinghub.elsevier.com/retrieve/pii/S0741521400903069
3. Dasgupta R, Patel M. Venous malformations. Semin Pediatr Surg [Internet]. Elsevier; 2014 August [cited 2014 Nov. 11]; 23(4):198-202. Available from: http://www.ncbi.nlm.nih.gov/pubmed/25241098
4. Garzon M C, Huang J T, Enjolras O, Frieden I J. Vascular malformations: Part I. J Am Acad Dermatol [Internet]. 2007 March [cited 2014 Oct. 28]; 56(3):353-70; quiz 371-4. Available from: http://www.ncbi.nlm.nih.gov/pubmed/17317485
5. McRae M Y, Adams S, Pereira J, Parsi K, Wargon O. Venous malformations: Clinical course and management of vascular birthmark clinic cases. Australas J Dermatol [Internet]. 2013 February [cited 2014 Nov. 17]; 54(1):22-30. Available from: http://www.ncbi.nlm.nih.gov/pubmed/23082725
6. Jolink F, Van Steenwijk R P, Van der Horst C M. “Consider Obstructive Sleep Apnea in Patients with Oropharyngeal Vascular Malformations.” J Cranio-Maxillo-Facial Surg [Internet]. Elsevier Ltd; 2015; Available from: http://dx.doi.org/10.1016/j.jcms.2014.11.016
7. Soblet J, Limaye N, Uebelhoer M, Boon L M, Vikkula M. Variable Somatic TIE2 Mutations in Half of Sporadic Venous Malformations. Mol Syndromol [Internet]. 2013 April [cited 2014 Nov. 13]; 4(4):179-83. Available from: http://www.pubmedcentral.nih.gov/articlerender.fcgi?artid=3666452&tool=pmcentrez&rendertype=abstract
8. Vikkula M, Boon L M, Carraway K L, Calvert J T, Diamonti a. J, Goumnerov B, et al. Vascular dysmorphogenesis caused by an activating mutation in the receptor tyrosine kinase TIE2. Cell [Internet]. Cell Press; 1996; 87(7):1181-90. Available from: http://dx.doi.org/10.1016/S0092-8674(00)81814-0
9. Steiner F, FitzJohn T, Tan S T. Ethanol sclerotherapy for venous malformation. ANZ J Surg. 2014; 1-6.
10. Steiner F, Fitzjohn T, Tan S T. Surgical treatment for venous malformation. J Plast Reconstr aesthetic Surg [Internet]. 2013 December [cited 2014 Nov. 12]; 66(12):1-9. Available from: http://www.ncbi.nlm.nih.gov/pubmed/24012651
11. Scherer K, Waner M. Nd:YAG lasers (1,064 nm) in the treatment of venous malformations of the face and neck: challenges and benefits. Lasers Med Sci [Internet]. 2007; 22(2):119-26. Available from: http://link.springer.com/10.1007/s10103-007-0443-0
12. Judith N, Ulrike E, Siegmar R, Matthias N, Jürgen H. Current concepts in diagnosis and treatment of venous malformations. J Craniomaxillofac Surg [Internet]. Elsevier Ltd; 2014 Apr. 1 [cited 2014 Nov. 11]; 42(7):1300-4. Available from: http://www.ncbi.nlm.nih.gov/pubmed/24969765
13. Lee B B, Bergan J J. Advanced management of congenital vascular malformations: a multidisciplinary approach. Cardiovasc Surg [Internet]. 2002 December; 10(6):523-33. Available from: http://www.ncbi.nlm.nih.gov/pubmed/12453680
14. van der Vleuten C J M, Kater A, Wijnen M H W A, Schultze Kool L I, Rovers M M. Effectiveness of Sclerotherapy, Surgery, and Laser Therapy in Patients With Venous Malformations: A Systematic Review. Cardiovasc Intervent Radiol [Internet]. 2013; 977-89. Available from: http://link.springer.com/10.1007/s00270-013-0764-2
15. Hyodoh H, Hori M, Akiba H, Tamakawa M, Hyodoh K, Hareyama M. Peripheral vascular malformations: imaging, treatment approaches, and therapeutic issues. Radiographics. 2005; 25 Suppl 1:S159-71.
16. Baek H J, Hong J P, Choi J W, Suh D C. Direct Percutaneous Alcohol Sclerotherapy for Venous Malformations of Head and Neck Region without Fluoroscopic Guidance: Technical Consideration and Outcome. Neurointervention [Internet]. 2011; 6(2):84-8. Available from: http://www.pubmedcentral.nih.gov/articlerender.fcgi?artid=3214818&tool=pmcentrez&rendertype=abstract
17. Albanese G, Kondo K L. Pharmacology of sclerotherapy. Semin Intervent Radiol [Internet]. 2010; 27(4):391-9. Available from: http://www.pubmedcentral.nih.gov/articlerender.fcgi?artid=3324200&tool=pmcentrez&rendertype=abstract
18. Puig S, Aref H, Chigot V, Bonin B, Brunelle F. Classification of venous malformations in children and implications for sclerotherapy. Pediatr Radiol [Internet]. 2003 February [cited 2014 Nov. 7]; 33(2):99-103. Available from: http://www.ncbi.nlm.nih.gov/pubmed/12557065
19. Hammer F D, Boon L M, Mathurin P, Vanwijck R R. Ethanol Sclerotherapy of Venous Malformations: Evaluation of Systemic Ethanol Contamination. J Vasc Interv Radiol [Internet]. 2001 May; 12(5):595-600. Available from: http://linkinghub.elsevier.com/retrieve/pii/S1051044307614821
20. Dubois J. Predominantly Venous Malformation. In: Golzarian J, Sun S, Sharafuddin M J, editors. Vascular Embolotherapy [Internet]. Berlin/Heidelberg: Springer-Verlag; 2006. p. 21-32. Available from: http://link.springer.com/10.1007/3-540-33257-X_2
21. Lee B B, Do Y S, Byun H S, Choo I W, Kim D I, Huh S H. Advanced management of venous malformation with ethanol sclerotherapy: Mid-term results. J Vasc Surg. 2003; 37(3):533-8.
22. In HL, Keon H K, Jeon P, Hong S B, Kim H J, Sung T K, et al. Ethanol sclerotherapy for the management of craniofacial venous malformations: The interim results. Korean J Radiol. 2009; 10(3):269-76.
23. Yakes W F, Luethke J M, Parker S H, Stavros A T, Rak K M, Hopper K D, et al. Ethanol embolization of vascular malformations. Radiographics [Internet]. 1990; 10(5):787-96. Available from: http://www.ncbi.nlm.nih.gov/pubmed/2217971
24. Fyhrquist F, Saijonmaa O. Renin-angiotensin system revisited. J Intern Med [Internet]. 2008 September [cited 2014 Oct. 29]; 264(3):224-36. Available from: http://www.ncbi.nlm.nih.gov/pubmed/18793332
25. Bemstein K E, Ong F S, Blackwell W B, Shah K H, Giani J F, Gonzalez-Villalobos R A, et al. A modern understanding of the traditional and nontraditional biological functions of angiotensin-converting enzyme. Pharmacol Rev [Internet]. 2013 January [cited 2014 Nov. 23]; 65(1):1-46. Available from: http://www.pubmedcentral.nih.gov/articlerender.fcgi?artid=3565918&tool=pmcentrez&rendertype=abstract
26. Lavoie J L, Sigmund C D. Minireview: overview of the renin-angiotensin system—an endocrine and paracrine system. Endocrinology [Internet]. 2003 June [cited 2014 Nov. 18]; 144(6):2179-83. Available from: http://www.ncbi.nlm.nih.gov/pubmed/12746271
27. Nguyen Dinh Cat A, Touyz R M. A new look at the renin-angiotensin system—focusing on the vascular system. Peptides [Internet]. 2011 October [cited 2014 Nov. 12]; 32(10):2141-50. Available from: http://www.ncbi.nlm.nih.gov/pubmed/21945916
28. Itinteang T, Tan S T, Brasch H, Day D J. Primitive mesodermal cells with a neural crest stem cell phenotype predominate proliferating infantile haemangioma. J Clin Pathol. 2010; 63(9):771-6.
29. Itinteang T, Brasch H, Tan S T, Day D J. Expression of components of the renin-angiotensin system in proliferating infantile haemangioma may account for the propranolol-induced accelerated involution. Journal of Plastic, Reconstructive and Aesthetic Surgery. 2011. p. 759-65.
30. Itinteang T, Davis P F, Tan S T. Tretment of Infantile Hemangioma with an ACE Inhibitor: A Paradigm Shift. In: Onuigbo M A, editor. ACE Inhibitors Volume 2. Nova Science Publishers, Inc.; 2014. p. 323-32.
31. Tan S T, Itinteang T, Day D J, O'Donnell C, Mathy J a, Leadbitter P. Treatment of infantile haemangioma with captopril. Br J Dermatol [Internet]. 2012 September [cited 2014 Nov. 11]; 167(3):619-24. Available from: http://www.ncbi.nlm.nih.gov/pubmed/22533490
32. Tan S T, Itinteang T, Leadbitter P. Low-dose propranolol for infantile haemangioma. J Plast Reconstr Aesthet Surg [Internet]. 2011 March; 64(3):292-9. Available from: http://dx.doi.org/10.1016/j.bjps.2010.06.010
33. Tan E M S, Itinteang T, Chudakova D A, Dunne J C, Marsh R, Brasch H D, et al. Characterisation of lymphocyte subpopulations in infantile haemangioma. J Clin Pathol [Internet]. 2015 October; 68(10):812-8. Available from: http://jcp.bmj.com/lookup/doi/10.1136/jclinpath-2015-203073
34. Nguyen G, Delarue F, Burcklé C, Bouzhir L, Giller T, Sraer J. Pivotal role of the renin/prorenin receptor in angiotensin II production and cellular responses to renin. J Clin Invest. 2002; 109(11): 1417-27.
35. Tissue expression of ACE—Staining in kidney—The Human Protein Atlas [Internet]. [cited 2015 December 10]. Available from: http://www.proteinatlas.org/ENSG00000159640-ACE/tissue/kidney
36. Zhuo J, Dean R, MacGregor D, Alcom D, Mendelsohn F A. Presence of angiotensin II AT2 receptor binding sites in the adventitia of human kidney vasculature. Clin Exp Pharmacol Physiol Suppl [Internet]. 1996; 3:5147-54. Available from: http://www.ncbi.nlm.nih.gov/pubmed/8993855
37. de Gasparo M, Catt K J, Inagami T, Wright J W, Unger T. International union of pharmacology. XXIII. The angiotensin II receptors. Pharmacol Rev [Internet]. 2000 September; 52(3):415-72. Available from: http://www.ncbi.nlm.nih.gov/pubmed/10977869
38. Dinh D T, Frauman A G, Johnston C I, Fabiani M E. Angiotensin receptors: distribution, signalling and function. Clin Sci [Internet]. 2001 May 1 [cited 2014 Dec. 5]; 100(5):481-92. Available from: http://www.clinsci.org/cs/100/cs1000481.htm
39. Deshayes F, Nahmias C. Angiotensin receptors: a new role in cancer? Trends Endocrinol Metab [Internet]. 2005 September [cited 2014 Nov. 19]; 16(7):293-9. Available from: http://www.ncbi.nlm.nih.gov/pubmed/16061390
40. Zambidis E T, Park T S, Yu W, Tam A, Levine M, Yuan X, et al. Expression of angiotensin-converting enzyme (CD143) identifies and regulates primitive hemangioblasts derived from human pluripotent stem cells. Blood [Internet]. 2008 Nov. 1 [cited 2014 Nov. 24]; 112(9):3601-14. Available from: http://www.pubmedcentral.nih.gov/articlerender.fcgi?artid=2572789&tool=pmcentrez&rendertype=abstract
41. Nguyen G, Danser a HJ. Prorenin and (pro)renin receptor: a review of available data from in vitro studies and experimental models in rodents. Exp Physiol [Internet]. 2008; 93(5):557-63. Available from: http://www.ncbi.nlm.nih.gov/pubmed/18376005
42. Abematsu T, Okamoto Y, Nakagawa S, Kurauchi K, Kodama Y, Nishikawa T, et al. Rectosigmoid colon venous malformation successfully treated with propranolol and celecoxib. J Pediatr Surg Case Reports [Internet]. Elsevier Inc; 2015; 3(8):331-3. Available from: http://linkinghub.elsevier.com/retrieve/pii/S2213576615000846
43. Holmer S R, Hengstenberg C, Mayer B, Engel S, Lowel H, Riegger G A J, et al. Marked suppression of renin levels by beta-receptor blocker in patients treated with standard heart failure therapy: a potential mechanism of benefit from beta-blockade. J Intern Med [Internet]. 2001 February; 249(2):167-72. Available from: http://doi.wiley.com/10.1046/j.1365-2796.2001.00786.x
44. Wang F, Lu X, Peng K, Zhou L, Li C, Wang W, et al. COX-2 mediates angiotensin II-induced (pro)renin receptor expression in the rat renal medulla. Am J Physiol Renal Physiol [Internet]. 2014; 307(1):F25-32. Available from: http://ajprenal.physiology.org/cgi/doi/10.1152/ajprenal.00548.2013
45. Mogler C, Beck C, Kulozik A, Penzel R, Schirmacher P, Breuhahn K. Elevated expression of c-kit in small venous malformations of blue rubber bleb nevus syndrome. Rare Tumors [Internet]. 2010 January [cited 2014 Dec. 13]; 2(2):e36. Available from: http://www.pubmedcentral.nih.gov/articlerender.fcgi?artid=2994504&tool=pmcentrez&rendertype=abstract
46. Otani A, Takagi H, Oh H, Koyama S, Honda Y. Angiotensin II induces expression of the Tie2 receptor ligand, angiopoietin-2, in bovine retinal endothelial cells. Diabetes. 2001; 50:867-75.
47. Eklund L, Olsen B R. Tie receptors and their angiopoietin ligands are context-dependent regulators of vascular remodeling. Exp Cell Res. 2006; 312:630-41.
48. Holash J, Wiegand S J, Yancopoulos G D. New model of tumor angiogenesis: dynamic balance between vessel regression and growth mediated by angiopoietins and VEGF. Oncogene. 1999; 18:5356-62.
49. Wassef M, Blei F, Adams D, et al. Vascular Anomalies Classification: Recommendations From the International Society for the Study of Vascular Anomalies. Pediatrics 2015; 136: e203.
50. Thompson L D. Head and Neck Pathology: A Volume in Foundations in Diagnostic Pathology Series. Elsevier Health Sciences; 2012: 28.
51. Kerr D A. Granuloma pyogenicum. Oral Surg, Oral Med, Oral Pathol 1951; 4: 158.
52. Lever W F, Elder D E, Elenitsas R, Johnson B L, Murphy G F. Lever's histopathology of the skin: Lippincott Williams & Wilkins; 2009.
53. Mills S E, Cooper P H, Fechner R E. Lobular capillary hemangioma: the underlying lesion of pyogenic granuloma. A study of 73 cases from the oral and nasal mucous membranes. Am J Surg Pathol 1980; 4: 470.
54. Patrice S J, Wiss K, Mulliken J B. Pyogenic Granuloma (lobular capillary hemangioma): A Clinicopathologic study of 178 cases. Pediatr Dermatol 1991; 8: 267.
55. Gupta A, Kozakewich H. Histopathology of vascular anomalies. Clin Plast Surg 2011; 38: 31-44.
56. Waraasawapati S, Koonmee S, Kusama H, Kudo M. Extramedullary hematopoiesis in pyogenic granuloma. Pathol Internatl 2013; 63: 492.
57. Sills E, Zegarelli D, Hoschander M, Strider W. Clinical diagnosis and management of hormonally responsive oral pregnancy tumor (pyogenic granuloma). J Reproduct Med 1996; 41: 467.
58. Harris M N, Desai R, Chuang T Y, Hood A F, Mirowski G W. Lobular capillary hemangiomas: An epidemiologic report, with emphasis on cutaneous lesions. J Am Acad Dermatol 2000; 42: 1012.
59. Gordón-Núñez M A, de Vasconcelos Carvalho M, Benevenuto T G, Lopes M F F, Silva L M M, Galvio H C. Oral pyogenic granuloma: A Retrospective analysis of 293 Cases in a Brazilian Population. J Oral Maxillofac Surg 2010; 68: 2185.
60. Choudhary S, MacKinnon C A, Morrissey G P, Tan S T. A case of giant nasal pyogenic granuloma gravidarum. The Journal of craniofacial surgery 2005; 16: 319.
61. Lee J, Sinno H, Tahiri Y, Gilardino M S. Treatment options for cutaneous pyogenic granulomas: a review. J Plast Reconstr Aesthet Surg 2011; 64: 1216.
62. Sud A R, Tan S T. Pyogenic granuloma-treatment by shave-excision and/or pulsed-dye laser. J Plast Reconstr Aesthet Surg 2010; 63: 1364.
63. Thomson J A, Itskovitz-Eldor J, Shapiro S S, et al. Embryonic stem cell lines derived from human blastocysts. Science 1998; 282: 1145.
64. Laslett A L, Filipczyk A A, Pera M F. Characterization and culture of human embryonic stem cells. Trends in Cardiovasc Med 2003; 13: 295.
65. Health NIo. NIH Human Embryonic Stem Cell Registry. Jul. 13, 2011 http://grants.nih.gov/stem_cells/registry/current.htm.
66. Nichols J, Zevnik B, Anastassiadis K, et al. Formation of pluripotent stem cells in the mammalian embryo depends on the pou transcription factor Oct4. Cell 1998; 95: 379.
67. Rodda D J, Chew J-L, Lim L-H, et al. Transcriptional regulation of nanog by OCT4 and SOX2. Journal Biol Chem 2005; 280: 24731.
68. Niwa H, Ogawa K, Shimosato D, Adachi K. A parallel circuit of LIF signalling pathways maintains pluripotency of mouse ES cells. Nature 2009; 460: 118.
69. Burdon T, Smith A, Savatier P. Signalling, cell cycle and pluripotency in embryonic stem cells. Trends in Cell Biol 2002; 12: 432.
70. Niwa H. How is pluripotency determined and maintained? Development 2007; 134: 635.
71. Avilion A A, Nicolis S K, Pevny L H, Perez L, Vivian N, Lovell-Badge R. Multipotent cell lineages in early mouse development depend on SOX2 function. Genes & Development 2003; 17: 126.
72. Boyer L A, Lee T I, Cole M F, et al. Core transcriptional regulatory circuitry in human embryonic stem cells. Cell 2005; 122: 947.
73. Episkopou V. SOX2 functions in adult neural stem cells. Trends in Neurosc 2005; 28: 219.
74. Shi W, Wang H, Pan G, Geng Y, Guo Y, Pei D. Regulation of the pluripotency marker Rex-1 by Nanog and Sox2. J Biol Chem 2006; 281: 23319.
75. Do D V, Ueda J, Messerschmidt D M, et al. A genetic and developmental pathway from STAT3 to the OCT4-NANOG circuit is essential for maintenance of ICM lineages in vivo. Genes & Development 2013; 27: 1378.
76. Yu J, Vodyanik M A, Smuga-Otto K, et al. Induced pluripotent stem cell lines derived from human somatic cells. Science 2007; 318: 1917.
77. Rowlands C G, Rapson D, Morell T. Extramedullary Hematopoiesis in a Pyogenic Granuloma. Am J Dermatopathol 2000; 22: 434-8.
78. Vega Harring S M, Niyaz M, Okada S, Kudo M. Extramedullary hematopoiesis in a pyogenic granuloma: a case report and review. J Cutan Pathol 2004; 31: 555.
79. Godfraind C, Calicchio M L, Kozakewich H. Pyogenic granuloma, an impaired wound healing process, linked to vascular growth driven by FLT4 and the nitric oxide pathway. Modern Pathol 2013; 26: 247.
80. Itinteang T, Tan S T, Brasch H D, et al. Infantile haemangioma expresses embryonic stem cell markers. J Clin Pathol 2012; 65: 394.
81. Tan E M, Chudakova D A, Davis P F, et al. Characterisation of subpopulations of myeloid cells in infantile haemangioma. J Clin Pathol 2015: jclinpath-2014-202846.
82. Markku M, Zeng-Feng W, Anders P, et al. ERG transcription factors as an immunohistochemical marker for vascular endothelial tumors and prostatic carcinoma. Am J Surg Pathol. 2011; 35: 432.
83. Chew J L, Loh Y H, Zhang W, et al. Reciprocal transcriptional regulation of Pou5f1 and Sox2 via the Oct4/Sox2 complex in embryonic stem cells. Mol Cell Biol 2005; 25: 6031.
84. Zeineddine D, Hammoud A A, Mortada M, Boeuf H. The Oct4 protein: more than a magic stemness marker. Am J Stem Cells 2014; 3: 74.
85. Chunhui X, Margaret S I, Jerrod D, et al. Feeder-free growth of undifferentiated human embryonic stem cells. Nature Biotechnol 2001; 19: 971.
86. Rodda D J, Chew J-L, Lim L-H, et al. Transcriptional regulation of nanog by OCT4 and SOX2. J Biol Chem 2005; 280: 24731.
87. Atlasi Y, Mowla S J, Ziaee S A M, Bahrami Ar. OCT□4, an embryonic stem cell marker, is highly expressed in bladder cancer. Internatl J Cancer 2007; 120: 1598.
88. Itinteang T, Tan S T, Brasch H D, et al. Infantile haemangioma expresses embryonic stem cell markers. J Clin Pathol 2012; 65: 394.
89. Itinteang T, Tan S T, Brasch H, Day D J. Primitive mesodermal cells with a neural crest stem cell phenotype predominate proliferating infantile haemangioma. J Clin Pathol 2010; 63: 771.
90. Tan E M, Itinteang T, Chudakova D A, Dunne J C, Marsh R, Brasch H D, et al. Characterisation of lymphocyte subpopulations in infantile haemangioma. J Clin Pathol. 2015; 68(10)812-8. doi:jclinpath-2015-203073.
91. Bradshaw A, Wickremesekera A, Brasch H D, Chibnall A M, Davis P F, Tan S T, et al. Cancer stem cells in glioblastoma multiforme. Front Surg. 2016; 3:48. doi:10.3389/fsurg.2016.00048
92. Baillie R, Itinteang T, Helen H Y, Brasch H D, Davis P F, Tan S T. Cancer stem cells in moderately differentiated oral tongue squamous cell carcinoma. J Clin Pathol. 2016; 69:742-744. doi:jclinpath-2015-203599.
93. Siljee S, Keane E, Marsh R, Brasch H D, Tan S T, Itinteang T. Expression of the Components of the Renin-Angiotensin System in Venous Malformation. Frontiers in Surgery. Front Surg. 2016; 3:24.
94. Soblet J, Limaye N, Uebelhoer M, Boon L, Vikkula M. Variable somatic TIE2 mutations in half of sporadic venous malformations. Mol Syndromol. 2013; 4(4):179-83.
95. McRae M Y, Adams S, Pereira J, Parsi K, Wargon O. Venous malformations: clinical course and management of vascular birthmark clinic cases. Australas J Dermatol. 2013; 54(1):22-30.4.
96. Garzon M C, Huang J T, Enjolras O, Frieden I J. Vascular malformations: part I. J Am Acad Dermatol. 2007; 56(3):353-70.
97. Boscolo E, Limaye N, Huang L, Kang K-T, Soblet J, Uebelhoer M, et al. Rapamycin improves TIE2-mutated venous malformation in murine model and human subjects. J Clin Invest. 2015; 125(9):3491-504.
98. Vikkula M, Boon L M, Iii K L C, Calvert J T, Diamonti A J, Goumnerov B, et al. Vascular dysmorphogenesis caused by an activating mutation in the receptor tyrosine kinase TIE2. Cell. 1996; 87(7):1181-90.
99. Jolink F, Van Steenwijk R, Van der Horst C. Consider obstructive sleep apnea in patients with oropharyngeal vascular malformations. J Cranio-Maxillofac Surg. 2015; 43(10):1937-41.
100. Li Z, Huang H, Boland P, Dominguez M G, Burfeind P, Lai K-M, et al. Embryonic stem cell tumor model reveals role of vascular endothelial receptor tyrosine phosphatase in regulating Tie2 pathway in tumor angiogenesis. Proc Natl Acad Sc. 2009; 106(52):22399-404.
101. Itinteang T, Tan S T, Brasch H D, Steel R, Best H A, Vishvanath A, et al. Infantile haemangioma expresses embryonic stem cell markers. J Clin Pathol. 2012; 65:394-398. doi: jclinpath-2011-200462.
102. Levy D E, Lee C-k. What does Stat3 do? J Clin Invest. 2002; 109(9):1143-8.
103. Chen H, Aksoy I, Gonnot F, Osteil P, Aubry M, Hamela C, et al. Reinforcement of STAT3 activity reprogrammes human embryonic stem cells to naive-like pluripotency. Nature Commun. 2015; 13; 6:7095. doi: 10.1038/ncomms8095
104. Annovazzi L, Mellai M, Caldera V, Valente G, Schiffer D. SOX2 expression and amplification in gliomas and glioma cell lines. Cancer Genomics-Proteomics. 2011; 8(3): 139-47.
105. Hao L, Zhao Y, Wang Z, Yin H, Zhang X, He T, et al. Expression and clinical significance of SALL4 and β-catenin in colorectal cancer. J Mol Histol. 2016; 47(2):117-28.
106. Zhao W, Ji X, Zhang F, Li L, Ma L. Embryonic stem cell markers. Molecules. 2012; 17(6):6196-236.
107. Boyer L A, Lee T I, Cole M F, Johnstone S E, Levine S S, Zucker J P, et al. Core transcriptional regulatory circuitry in human embryonic stem cells. cell. 2005; 122(6):947-56.
108. Pan G, Thomson J A. Nanog and transcriptional networks in embryonic stem cell pluripotency. Cell Res. 2007; 17(1):42-9.
109. Mogler C, Beck C, Kulozik A, Penzel R, Schirmacher P, Breuhahn K. Elevated expression of c-kit in small venous malformations of blue rubber bleb nevus syndrome. Rare Tumors. 2010; 2(2):36.
110. Nguyen G. Renin, (pro) renin and receptor: an update. Clin Sc. 2011; 120(5):169-78.
111. 34. Sato N, Meijer L, Skaltsounis L, Greengard P, Brivanlou A H. Maintenance of pluripotency in human and mouse embryonic stem cells through activation of Wnt signaling by a pharmacological GSK-3-specific inhibitor. Nature Med. 2004; 10(1):55-63.

1. A method for preventing, treating, or managing vascular anomalies in a patient in need thereof, the method comprising administering a therapeutic agent(s) to the patient in an amount sufficient to selectively eradicate, or inhibit the growth, proliferation and/or differentiation of embryonic stem cells associated with vascular anomalies, wherein the embryonic stem cells are characterized by (i) the expression of one or more embryonic stem cell biomarkers, and (ii) the expression of one or more biomarkers associated with the Renin-Angiotensin System.

2. The method according to claim 1, wherein the embryonic stem cells are characterized by expression of one or more embryonic stem cell biomarkers selected from the group consisting of Cripto, ABCG2, Alkaline Phosphatase/ALPL, CD9, FGF-4, GDF-3, Integrin alpha 6/CD49f, Integrin beta 1/CD29, NANOG, OCT3/4, Podocalyxin, SOX2, SSEA-3, SSEA-4, STAT3, SSEA-1, FoxD3, DPPA5/ESG1, Rex-1/ZFP42, DPPA4, LIN-28A, UTF1, Lefty-A, Lefty-1, TBX3, ESGP, TRA-1-60(R), TRA-1-81, 5T4, TBX2, ZIC3, CD30/TNFRSF8, KLF5, c-Myc, GCNF/NR6A1, SUZ12, Smad2, CDX2, TROP-2, CD117/c-kit, LIN-41, Integrin alpha 6 beta 4, THAP11, Smad2/3, TBX5, TEX19, OCT4A, TEX19.1, DPPA2, Activin RIB/ALK-4, Activin RIIB, FGF-5, GBX2, Stella/Dppa3, DNMT3B, F-box protein 15/FBXO15, LIN-28B, Integrin alpha 6 beta 1, KLF4, ERR beta/NR3B2, EpCAM/TROP1, TERT, CHD1, Cbx2, c-Maf and L1TD1.

3. The method according to claim 1, wherein the embryonic stem cells are characterized by expression of one or more embryonic stem cell biomarkers selected from the group consisting of OCT4, SOX2, NANOG and PSTAT3.

4. The method according to claim 1, wherein the embryonic stem cells are characterised by expression of the embryonic stem cell biomarkers consisting in SOX2, NANOG and PSTAT3.

5. The method according to claim 1, wherein the embryonic stem cells are characterized by expression of one or more Renin-Angiotensin System biomarkers selected from the group consisting of Renin Receptor, Angiotensin Converting Enzyme, Angiotensin II Receptor 1, Angiotensin II receptor 2, a soluble form of the Renin Receptor and a soluble form of Angiotensin Converting Enzyme.

6. The method according to claim 1, wherein vascular anomalies is selected from the group consisting of pyogenic granuloma, venous malformation, infantile hemangioma, lymphatic malformation, capillary malformation, arterial malformation, arterio-venous malformation, lymphatico-venous malformation, capillary-venous malformation, capillary-lymphatico-venous malformation, kaposiform hemangioendotheloima, tufted angioma, hepatic hemangioendothelioma, and verrucous hemangioma.

7. The method according to claim 6, wherein vascular anomalies is venous malformation or pyogenic granuloma.

8. The method according to claim 1, wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

9. A method for preventing, treating, or managing vascular anomalies in a patient in need thereof, the method comprising administering a therapeutic agent to the patient in an amount sufficient to selectively eradicate or inhibit the growth, proliferation and/or differentiation of embryonic stem cells associated with vascular anomalies, wherein the embryonic stem cells are characterized by (i) expression of the embryonic stem cell biomarkers OCT4, SOX2, NANOG and PSTAT3, and (ii) expression of the Renin-Angiotensin System biomarkers Renin Receptor and Angiotensin Converting Enzyme, and wherein the therapeutic agent in selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.

10.-13. (canceled)

14. A kit for use in the treatment of vascular anomalies, the kit comprising a therapeutic agent sufficient to selectively eradicate, or inhibit the growth, proliferation and/or differentiation of embryonic stem cells associated with vascular anomalies, together with instructions for how to administer a therapeutic dose to the subject.

15. The kit according to claim 14, wherein the therapeutic agent is selected from the group consisting of Direct Renin Inhibitors (DRIs), Angiotensin-Converting Enzyme Inhibitors (ACEIs), Angiotensin Receptor Blockers (ARBs), Beta-Blockers, Cyclo-oxygenase 2 Inhibitors, Chymase Inhibitors, Inhibitors of Cathepsin B, Cathepsin D and Cathepsin G, Calcium, Vitamin D, and Calcium Channel Blockers.
